(19) United States
(12) Patent Application Publication
(10) Pub. No.: US 2010/0042216 A1
(43) Pub. Date: Feb. 18, 2010

(54) Implant for Deploying Bone Graft Material and Methods Thereof

(75) Inventors: Thomas S. Kilpela, Marquette, MI (US); Jeffrey A. Hoffman, Marquette, MI (US); Brian P. Janowski, Marquette, MI (US)

Correspondence Address: FITCH EVEN TABIN & FLANNERY, 120 SOUTH LASALLE STREET, SUITE 1600, CHICAGO, IL, 60603-3406, US

(73) Assignee: PIONEER SURGICAL TECHNOLOGY, INC., Marquette, MI (US)

(21) Appl. No.: 12/542,548

(22) Filed: Aug. 17, 2009

Related U.S. Application Data
(60) Provisional application No. 61/089,138, filed on Aug. 15, 2008.

(51) Int. Cl. A61F 2/44

(52) U.S. Cl. 623/17.11

(57) ABSTRACT

Invertebral implants are provided for carrying bone graft material and extruding the bone graft material between the implant and the adjacent vertebral bodies. The implants include first and second implant extrusion members shiftable relative to one another between a pre-implantation configuration having an enlarged cavity for receiving bone graft material therein and an implantation configuration having a smaller cavity. As the first and second implant extrusion members shift during implantation to the implantation configuration bone graft material is extruded from the cavity and into gaps or spaces between the upper and lower vertebral engaging surfaces of the implant and the adjacent vertebral bodies. After extrusion the bone graft material is subjected to spinal loading and stress to encourage strong bone growth therethrough.

### CROSS REFERENCE TO RELATED APPLICATIONS

This application claims the benefit of the filing date of U.S. Provisional Application 61/089,138, filed Aug. 15, 2008, which is hereby incorporated in its entirety herein.

### FIELD OF THE INVENTION

The invention relates to implant devices carrying bone graft material for implantation within an intervertebral space for immobilization and fusion of adjacent vertebrae.

### BACKGROUND OF THE INVENTION

Back pain can develop due to traumatic injury to the spine, disease, or genetic defect. Typically, damage to the spine can cause the vertebral discs to bulge or herniate, which can in turn impinge on the nerves of the spine. One method of treating a damaged disc is by immobilizing the area around the injured portion and fusing the immobilized portion by promoting bone growth between the immobilized spine portions. This often requires implantation of an intervertebral device to provide the desired spacing between adjacent vertebrae.

Typically, prior to implantation, all or a portion of the injured intervertebral disc is excised to provide a space for implanting the intervertebral implant device. In general, vertebral endplates are slightly concave, with concavity varying from endplate to endplate. In particular, superior endplates tend to be less concave than inferior endplates. As a result, the upper and lower support surfaces of existing intervertebral implants typically do not exactly match the contour of the superior and inferior surfaces, or endplates, of the adjacent vertebrae. In addition, even with implants configured to generally conform to the surfaces of the vertebral end plates, the lack of uniformity of the surfaces of vertebral end plates from vertebrae to vertebrae and patient to patient can result in gaps between the endplates and the vertebral engaging surfaces of the implant.

Intervertebral devices can be made of any suitable materials which are compatible with the uses and environments of the human body. In particular, the implant devices are generally constructed so as to be nonreactive and non-antigenic to biological systems, i.e. bio-compatible. Exemplary metallic materials include titanium, stainless steel, Nitinol (Nickel Titanium Naval Ordinance Laboratory) or other metal alloys. Alternatively, implants have been constructed from polyetheretherketone (PEEK) or any polymer of the poly-aryl-ether-ketone family such as, but not limited to, /epoly-ether-ketone (PEK) and poly-ether-ketone-ether-ketone-ketone (PEKEKK). The polymer materials can be machined or formed from injection molding to various configurations. Further, the implant devices can be coated or otherwise treated to promote bone growth or fusion therewith.

Bone graft material carried by implants has been used to promote bone growth between adjacent vertebrae for fusion thereof. The bone graft material instigates and promotes natural bone growth, which in turn fuses the adjacent vertebrae thereby providing permanent structural support therebetween.

Bone graft material can be autograft, allograft and/or artificial material. With autograft and allograft, the bone can be used whole or can be comminuted and combined with other materials, such as blood products and bone marrow, to form a mixture. Depending on the use, the mixture can have varying consistencies and viscosities. Exemplary consistencies include putty-like, paste-like and syrup-like.

Exemplary artificial bone graft material includes calcium phosphates (such as hydroxyapatite and tri-calcium phosphate). Other suitable materials includes those described in U.S. Pat. No. 6,013,591, J. Y. Ying, E. S. Ahn, and A. Nakahira, “Nanocrystalline apatites and composites, prostheses incorporating them, and method for their production,” which is incorporated by reference in its entirety herein. Another exemplary material is described in U.S. Pat. No. RE39,196 E, Jackie Y. Ying, Edward S. Ahn, and Atsushi Nakahira, “Nanocrystalline apatites and composites, prostheses incorporating them, and method for their production,” which is incorporated by reference in its entirety herein.

Another exemplary product is described in U.S. Pat. No. 6,231,881 B1, Anton-Lewis Usala, and Richard Chris Klann, “Medium and matrix for long-term proliferation of cells,” which is incorporated by reference in its entirety herein; and U.S. Pat. No. 6,730,315 B2, Anton-Lewis Usala, and Richard Chris Klann, “Medium and matrix for long-term proliferation of cells,” which is incorporated by reference in its entirety herein; and U.S. Pat. No. 6,315,994 B2, Anton-Lewis Usala, and Richard Chris Klann, “Medium and matrix for long-term proliferation of cells,” which is incorporated by reference in its entirety herein. Similarly, U.S. Pat. No. 6,713, 079 B2, U.S. Pat. No. 6,261,587 B1, U.S. Pat. No. 5,824,331, U.S. Pat. No. 6,068,974, U.S. Pat. No. 6,352,707 B1, U.S. Pat. No. 6,270,977 B1, U.S. Pat. No. 5,614,205, U.S. Pat. No. 6,790,455 B2, and U.S. Pat. No. 5,922,339 and U.S. Patent Application 2005/0118230 A1, Ronald Stewart Hill, Richard Chris Klann, and Francis V. Lambert, “Methods and compositions for regenerating connective tissue,” are incorporated by reference in their entirety herein.

Further exemplary artificial bone graft materials are sold by Pioneer Surgical Technologies, Inc., under the trade names E-Matrix, TrioMatrix, Nanoss and FortrOss.

For bone growth to occur, it is desirable for the bone and bone graft material to be exposed to stress. To promote healthy bone growth, some prior art implants attempt to control the stress placed on the bone graft material to allow for bone growth to occur in a controlled stress environment. In particular, these implants attempt to promote bone growth without a danger of overloading the newly formed bone by exposing the bone graft material to controlled amounts of stress.

For example, U.S. Pat. No. 6,395,035 to Bresina et al. discloses an implant having slots or openings in the implant outer, annular implant wall to control the amount of stress applied to the bone graft material within an implant cavity by allowing controlled compression of the implant. The slots are configured and positioned to provide different spring or resistance rates to allow the implant to be compressed upon spinal loading. In particular, the implant slots are configured to provide rapid compression of the implant up to a specified low stress load. However, in the presence of higher stress loading the implant provides higher resistance levels to implant compression so that the higher loading on the implant only creates small additional strain on the graft material. By way of the slotted construction of the annular wall of the implant body, the Bresina et al. implant attempts to control the amount of stress applied to the bone graft material. Since the amount of stress placed on the bone graft material for a given spinal loading is determined by the physical characteristics of the implant body, the subsequent bone growth is dependent on the loading and spring resistance assumptions made prior to the implantation of the implant.

However, it is believed that precision control over the stress to which the bone graft material will be exposed will be difficult if not impossible to achieve as this will largely depend on specifics unique to each patient. In addition, the volume of the bone graft material can change as it is exposed to different loading since with increased loading the bone graft material can be forced into and/or escape through the slots. With the slots filled with bone graft material, they will not provide the anticipated compression levels, and with the bone graft material in the slots or escaping out therefrom, the bone graft material may not be at the interface between the vertebrae and the implant.

In another approach, PCT Application No. PCT/US2008/085831 to Richelsoph discloses an implant which provides for a gradual shifting of spinal loading from the implant body to the bone graft material to attempt to control the stress applied to the bone graft material over time and thereby avoid damage during bone growth. In particular, the implant body includes an upper section, a lower section and chambers for being filled with bone graft material. The sections have a generally stepped wall annular configuration with the lower section configured to receive an inner, annular flange portion depending from the upper section. The lower section includes an inner, annular ledge portion at the bottom thereof on which at least one bioresorbable spacer is placed.

Additionally, radially extending throughbores extend through the annular wall of the lower section above the ledge portion thereof. The throughbores provide communication between the bone graft chamber in which the resorbable spacer is supported on the ledge portion and bodily fluids outside the implant body. The throughbores thus provide a flow path for the bodily fluids into the bone graft chamber formed by the annular sections of the implant body for contacting the resorbable spacers thereon. The spacer is configured to be resorbed by the by the bodily fluids over time, leading to a controlled reduction in size of the spacer during resorbtion. As a result of the decreased spacer size, the flange portion of the upper section gradually shifts axially further into the lower section chamber resulting in an overall height reduction of the implant body.

Upon insertion in the intervertebral space, the implant maintains an insertion height, even in the presence of a gap between the bone graft material and the vertebrae. Thus, during implantation the bone graft material remains in the chamber and is not intended to be forced out axially therefrom as the bone graft material would be exposed to stress earlier than desired. As the spacer resorbs over time and reduces in size, the flange of the upper section shifts further downward into the chamber of the lower section resulting in a reduced implant height. According to Richelsoph, this gradual reduction in height results in increased loading being applied to the bone graft material and newly formed bone growth. Further, Richelsoph asserts that the rate of resorbtion of the spacer and the corresponding reduction in implant body axial height is configured to correspond to the rate of bone growth through the implant chamber. More particularly, loading on the bone graft material due to the implant body height reduction is configured to be gradually increased and correspond to additional bone growth to handle the additional stress without damage thereto. Eventually, the spacer is fully resorbed and the axial height of the implant body is fully reduced such that the vertebrae are no longer supported by the implant body, but instead are supported by the bone graft material and/or the new bone growth extending between the vertebrae.

However, as stated above, is believed that precision control over the stress to which the bone graft material will be exposed will be difficult if not impossible to achieve as this will largely depend on specifics unique to each patient.

### SUMMARY OF THE INVENTION

An implant that carries bone graft material therein is provided and is configured to extrude bone graft material out therefrom during implantation. More particularly, the preferred implant includes first and second implant extrusion members that are operable to shift relative to each other from a pre-implantation configuration to an implantation configuration during implantation of the implant into an intervertebral space. The shifting of the extrusion members to the implantation configuration causes bone graft material in a cavity of the implant to be extruded out therefrom into the areas between upper and lower surfaces of the implant and corresponding facing surfaces of adjacent upper and lower vertebrae. In this manner, the bone graft material is immediately subjected to stress and loading after implantation. It is believed that such immediate exposure will beneficially improve the fusion between the vertebrae.

Generally herein it is thought that bone graft material will promote fusion best when exposed to stress such as generated during use with the implant inserted into the intervertebral space. Rather than attempting to control the stress that the bone graft material is exposed to, the present implant is configured to expose the bone graft material to stress immediately upon insertion of the implant into the intervertebral space and shifting of the implant body to its implantation configuration as by either the use of compression force from the distracted vertebrae when the distraction force on the vertebrae is released during the implantation procedure, or the use of a tool that shifts a portion of the implant body relative to another portion thereof.

Instead of attempting to control stress on the bone graft material, it is preferred that relative bending motion between the vertebrae can be controlled so as to avoid “pull-back” between the vertebrae. When deemed to be advantageous or otherwise necessary to promote fusion between adjacent vertebrae, rather than utilizing the implant itself, it is believed that relative bending motion between the vertebrae is better controlled by the use of additional intervertebral spacing connection devices, such as pedicle screw and rod systems and dynamized plates, rather than by complexly configured implant bodies or implants relying on biological activity for such purpose.

In particular, the intervertebral spacing connection devices can be used to avoid fore-and-aft or lateral bending of the spine between the vertebral bodies and between which the implant is implanted. Maintaining the relative vertical positions of the vertebral bodies reduces the occurrence of pull-back, which happens when the spine bends. For example, with forward bending, the forward portions of the facing vertebral surfaces will be shifted toward one another and the rearward portions of the same vertebral surfaces will be shifted away from one another. In this instance the rearward portions of the vertebral bodies will pull-back from the corresponding rear area of the implant body while compressing the forward area. Therefore, maintaining the relative vertical position of the vertebral bodies to be fused and reducing the occurrence of pull-back provides the constant loading which is beneficial to promoting bone growth. Nevertheless, it will be appreciated that the present implant need not be utilized with additional intervertebral spacing devices, particularly where used in area of the spine where “pull-back” may be of less concern.

As a result, the implant herein preferably does not act to provide a cushion or limit the amount of stress applied to the bone graft material. More particularly, the implant does not attempt to control the stresses applied to the bone graft material by providing controlled compression of the implant body. Instead, during and after implantation the bone graft material is subjected to the same stresses as the implant, thereby encouraging strong bone graft to withstand such loading.

In a preferred form, the implant includes upper and lower vertebral engaging surfaces for engaging facing surfaces of adjacent vertebral bodies. Each of the vertebral engaging surfaces includes an opening therein with the cavity extending between the openings. The vertebral engaging surface openings are configured to permit bone graft material to be extruded therethrough into the spaces between the vertebral engaging surfaces and the vertebral bodies as the implant extrusion members shift to the implantation configuration.

The implant further includes wall portions around the cavity for retaining the bone graft material in the cavity and forcing the extruded bone graft material toward the adjacent vertebral endplates. The wall portions lack any unobstructed openings therein while the first and second implant extrusion members are being shifted from the pre-implantation to the implantation configuration. Therefore, any openings in the wall portions, such as socket openings for being engaged by an insertion tool, are obstructed while the first and second implant extrusion members are shifted to the implantation configuration. As a result, the extruded bone graft material is directed to the desired areas at the interface between the implant and the vertebral endplates.

Optionally, bone graft material can be applied to the implant body after the first and second implant extrusion members have been shifted to the implantation configuration. In particular, bone graft material can be used to plug any socket openings in the wall portions. Further, bone graft material can be applied to the outer surface of the wall portions after the implant has been implanted between the adjacent vertebrae.

The first and second implant extrusion members can be shifted from their pre-implantation configuration to their implantation configuration in a variety of ways. For example, the extrusion members can be shifted vertically or axially relative to each other generally along the spinal axis or they can be shifted laterally relative to each other. The extrusion members can be provided with a ratchet connection for guiding the vertical or lateral shifting thereof.

In one form, the implant includes a ratchet connection for shifting the implant vertically generally along the spinal axis. The first implant extrusion member includes a flange having an upper vertebral engaging surface, an upper opening and a lower surface thereof, a wall portion depending from the lower surface of the flange about the upper opening and defining an upper cavity. The second implant extrusion member includes a lower vertebral engaging surface having a lower opening, an upper surface and a wall portion extending therebetween and about a lower cavity. The wall portions of the first and second implant extrusion members further have a ratchet connection therebetween defining the pre-implantation and implantation configurations. In the pre-implantation configuration the upper and lower cavities are packed with bone graft material and placed between adjacent vertebral bodies. The distraction tool is then removed, allowing the vertebral bodies to engage the upper and lower vertebral engaging surfaces of the implant body. The compressive forces applied by the vertebral bodies to the first and second implant extrusion members causes the first and second implant extrusion members to shift along the ratchet connection to the implantation configuration. The reduction in size of the implant cavity causes bone graft material within the implant cavity to be extruded therefrom and into spaces between the upper and lower vertebral engaging surfaces and the faces of the vertebral bodies. As a result, the bone graft material is in immediate contact with the vertebral endplates and is exposed to the same loading as the implant body.

In another form, the implant includes a ratchet connection for shifting the implant laterally between the adjacent vertebrae from the pre-implantation configuration to the implantation configuration. The first implant extrusion member includes an arcuate wall portion and pawl portions extending from either end. The second implant extrusion member includes an arcuate wall portion having recesses in either end corresponding to and configured to receive the pawl portion therein and provide a ratchet connection therewith. In the pre-implantation configuration, the pawl portions are partially received in the recesses of the second implant extrusion member. Additionally, the wall portions of the first and second implant extrusion members cooperate to form an implant cavity for being packed with bone graft material. With the cavity packed with bone graft material, the implant is positioned between adjacent vertebrae. A tool is used to shift the first and second implant extrusion members relative to one another to the implantation configuration, defined by the pawl portions of the first implant extrusion member being fully received in the recesses of the second implant extrusion member. Shifting the implant extrusion members to the implantation configuration decreases the implant cavity volume such that bone graft material is extruded therefrom into spaces between the upper and lower vertebral engagement surfaces and the vertebral bodies. As a result, the bone graft material is in immediate contact with the vertebral endplates and is exposed to the same loading as the implant body.

In an alternative configuration, the first implant extrusion member includes a wall portion extending around a cavity for receiving bone graft material therein. The second implant extrusion member includes a plug member for being inserted into the implant cavity. To receive the second implant extrusion member the wall portion of the first implant extrusion member includes a throughbore therein. The first and second implant extrusion members define the implantation configuration with the second implant extrusion member fully received by the first implant extrusion member. The pre-implantation configuration includes any relative position of the first and second implant extrusion members but the implantation configuration.

With the first and second implant extrusion members in the pre-implantation configuration, the cavity is packed with bone graft material and inserted between adjacent vertebrae. After the upper and lower vertebral engaging surfaces have been engaged by the vertebral bodies, the second implant extrusion member is shifted through the throughbore of the first implant extrusion member to the implantation configuration. As a result, the volume within the cavity decreases and bone graft material is extruded therefrom into spaces between the upper and lower vertebral engagement surfaces and the vertebral bodies. As a result, the bone graft material is in immediate contact with the vertebral endplates and is exposed to the same loading as the implant body.

### BRIEF DESCRIPTION OF THE DRAWINGS

FIG. 1 is an exploded perspective view of an implant in accordance with another aspect of the invention;

FIG. 2 is an exploded perspective view of the implant of FIG. 1 showing the first and second implant extrusion members adjacent to one another;

FIG. 3 is a perspective view of the implant of FIG. 1 in the pre-implantation configuration;

FIG. 4 is a perspective view of the implant of FIG. 1 in the implantation configuration;

FIG. 5 is an exploded end elevational view of the implant of FIG. 1 showing the center sleeve portion of the upper member;

FIG. 6 is an end elevational view of the implant of FIG. 1 in the pre-implantation configuration;

FIG. 7 is an end elevational view of the implant of FIG. 1 in the implantation configuration;

FIG. 8 is an exploded sectional end elevational view of the implant of FIG. 1 showing the central wall and sleeve portion of the extrusion members;

FIG. 9 is a sectional end elevational view of the implant of FIG. 1 in the pre-implantation configuration showing the engagement of the central wall portion of the second extrusion member and the sleeve portion of the first extrusion member;

FIG. 10 is a sectional end elevational view of the implant of FIG. 1 in the implantation configuration;

FIG. 11 is an exploded side elevational view of the implant of FIG. 1;

FIG. 12 is a side elevational view of the implant of FIG. 1 in the pre-implantation configuration;

FIG. 13 is a side elevational view of the implant of FIG. 1 in the implantation configuration;

FIG. 14 is an exploded sectional side elevational view of the implant of FIG. 1 showing the ratchet connection;

FIG. 15 is a sectional side elevational view of the implant of FIG. 1 in the pre-implantation configuration;

FIG. 16 is a sectional side elevational view of the implant of FIG. 1 in the implantation configuration;

FIG. 17 is a top view of the implant of FIG. 1;

FIG. 18 is a top sectional view of the implant of FIG. 1;

FIG. 19 is a bottom view of the implant of FIG. 1;

FIG. 20 is an exploded perspective view of a first embodiment of the implant and the bone graft material to be received in the implant cavity;

FIG. 21 is a perspective view of the implant of FIG. 20 in the pre-implantation configuration showing the bone graft material in the implant cavity;

FIG. 22 is a perspective view of the implant of FIG. 20 in the implantation configuration showing the bone graft material extruded out the opening in the upper vertebral engaging surface-;

FIG. 23 is an end elevational view of the implant of FIG. 20 in the pre-implantation configuration;

FIG. 24 is an end elevational view of the implant of FIG. 20 in the implantation configuration showing the bone growth material extending beyond the upper and lower surfaces of the implant body;

FIG. 25 is a sectional end elevational view of the implant of FIG. 20 in the pre-implantation configuration showing the bone graft material between the upper and lower surfaces and extending along the height of the implant and the engagement of the upper and lower members;

FIG. 26 is a sectional end elevational view of the implant of FIG. 20 in the implantation configuration showing the bone graft material extending beyond the upper and lower surfaces of the implant;

FIG. 27 is a side elevational view of the implant of FIG. 20 in the pre-implantation configuration showing the distance the flange of the first extrusion member extends above the upper surface of the second extrusion member;

FIG. 28 is a side elevational view of the implant of FIG. 20 in the implantation configuration showing the bone graft material extending beyond the upper and lower surfaces of the implant;

FIG. 29 is a side sectional view of the implant of FIG. 20 in the pre-implantation configuration showing a wedge portion of the first implant extrusion member engaging cantilever portions of the second implant extrusion member;

FIG. 30 is a side sectional view of the implant of FIG. 20 in the implantation configuration showing the wedge portion of the first implant extrusion member engaging and causing the distal ends of the cantilever portions of the second implant extrusion member to deflect downwardly;

FIG. 31 is a perspective view of the implant of FIG. 20 in the pre-implantation configuration and the insertion tool disengaged from the implant;

FIG. 32 is a perspective view of the implant of FIG. 20 with the insertion tool engaged with the implant in the pre-implantation configuration;

FIG. 33 is a perspective view of the implant of FIG. 20 in the implantation configuration with the tool disengaged from the implant;

FIG. 34 is a top view of the implant of FIG. 20 with the inserter tool disengaged from the implant;

FIG. 35 is a top view of the implant of FIG. 20 with the inserter tool engaged with the implant;

FIG. 36 is a top sectional view of the implant of FIG. 20 with inserter tool disengaged from the implant;

FIG. 37 is a top sectional view of the implant of FIG. 20 with the inserter tool engaged with the implant;

FIG. 38 is an enlarged top sectional view of the implant of FIG. 20 with inserter tool engaged with the implant;

FIG. 39 is an exploded view of an implant in accordance with another aspect of the invention;

FIG. 40 is a perspective view of the implant of FIG. 39 showing the wall opening therein for receiving the plug member;

FIG. 41 is a perspective view of the implant of FIG. 39 with the plug member inserted therein;

FIG. 42 is a front elevational view of the implant of FIG. 39 showing the wall opening;

FIG. 43 is a front elevational view of the implant of FIG. 39 with the plug member inserted in the wall opening;

FIG. 44 is a front sectional elevational view of the implant of FIG. 39 without the plug member inserted;

FIG. 45 is a front sectional elevational view of the implant of FIG. 39 with the plug member inserted;

FIG. 46 is a side elevational view of the implant of FIG. 39;

FIG. 47 is a side elevational view of the implant of FIG. 39 with the plug member inserted therein;

FIG. 48 is a sectional side elevational view of the implant of FIG. 39 without the plug member inserted;

FIG. 49 is a sectional side elevational view of the implant of FIG. 39 with the plug member inserted;

FIG. 50 is a top view of the implant of FIG. 39 without the plug member inserted;

FIG. 51 is a top view of the implant of FIG. 39 with the plug member inserted;

FIG. 52 is a top sectional view of the implant of FIG. 39 without the plug member inserted;

FIG. 53 is a top sectional view of the implant of FIG. 39 with the plug member inserted;

FIG. 54 is an end elevational view of the implant of FIG. 39 without the plug member inserted;

FIG. 55 is an end elevational view of the implant of FIG. 39 with the plug member inserted;

FIG. 56 is a back sectional elevational view of the implant of FIG. 39 without the plug member inserted;

FIG. 57 is a back sectional elevational view of the implant of FIG. 39 with the plug member inserted;

FIG. 58 is a perspective view of the implant of FIG. 39 implanted in the lumbar portion of a human spine;

FIG. 59 is an exploded perspective view of an implant in accordance with another aspect of the invention;

FIG. 60 is an exploded perspective view of the implant of FIG. 59 showing the first and second implant extrusion members adjacent one another;

FIG. 61 is a perspective view of the implant of FIG. 59 in the pre-implantation configuration;

FIG. 62 is a perspective view of the implant of FIG. 59 in the implantation configuration;

FIG. 63 is an exploded side elevational view of the implant of FIG. 59;

FIG. 64 is a side elevational view of the implant of FIG. 59 in the pre-implantation configuration;

FIG. 65 is a side elevational view of the implant of FIG. 59 in the implantation configuration;

FIG. 66 is a sectional side elevational view of the implant of FIG. 59 in the pre-implantation configuration;

FIG. 67 is a sectional side elevational view of the implant of FIG. 59 in an intermediate configuration;

FIG. 68 is a sectional side elevational view of the implant of FIG. 59 in the implantation configuration;

FIG. 69 is an exploded top view of the implant of FIG. 59;

FIG. 70 is a top view of the implant of FIG. 59 in the pre-implantation configuration;

FIG. 71 is a top view of the implant of FIG. 59 in the implantation configuration;

FIG. 72 is an exploded top sectional view of the implant of FIG. 59;

FIG. 73 is a top sectional view of the implant of FIG. 59 in the pre-implantation configuration;

FIG. 74 is a top sectional view of the implant of FIG. 59 in the implantation configuration;

FIG. 75 is a front elevational view of the implant of FIG. 59;

FIG. 76 is a perspective view of an implant in accordance with another aspect of the invention;

FIG. 77 is a top view of the implant of FIG. 76 in the implantation configuration showing the pre-implantation configuration of the wall portion in phantom;

FIG. 78 is a top sectional view of the implant of FIG. 76;

FIG. 79 is a side elevational view of the implant of FIG. 76;

FIG. 80 is a side sectional view of the implant of FIG. 76;

FIG. 81 is a perspective view of an implant in accordance with another aspect of the invention;

FIG. 82 is a top view of the implant of FIG. 81 showing the movement path of the inner wall in phantom;

FIG. 83 is a top sectional view of the implant of FIG. 81 showing the socket openings of the first and second implant extrusion members;

FIG. 84 is a side elevational view of the implant of FIG. 81;

FIG. 85 is a sectional side elevational view of the implant of FIG. 81; and

FIG. 86 is a perspective view of an insertion tool.

### DETAILED DESCRIPTION OF THE PREFERRED EMBODIMENTS

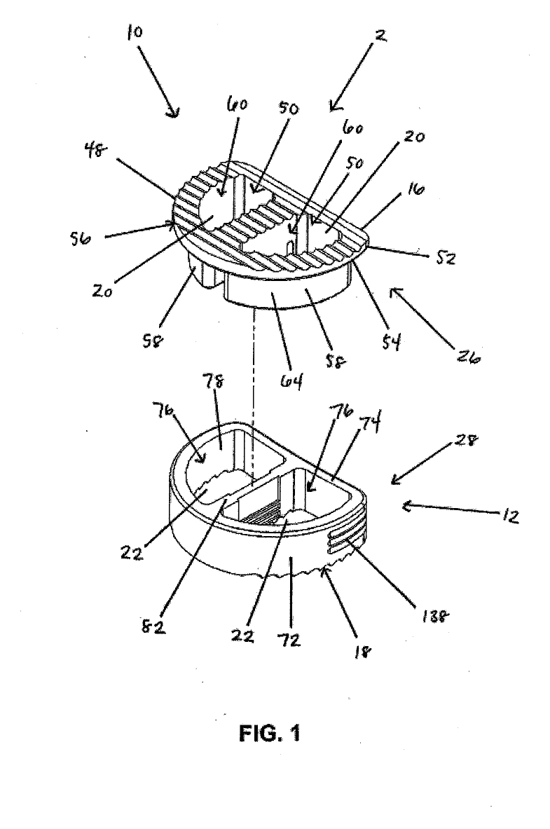

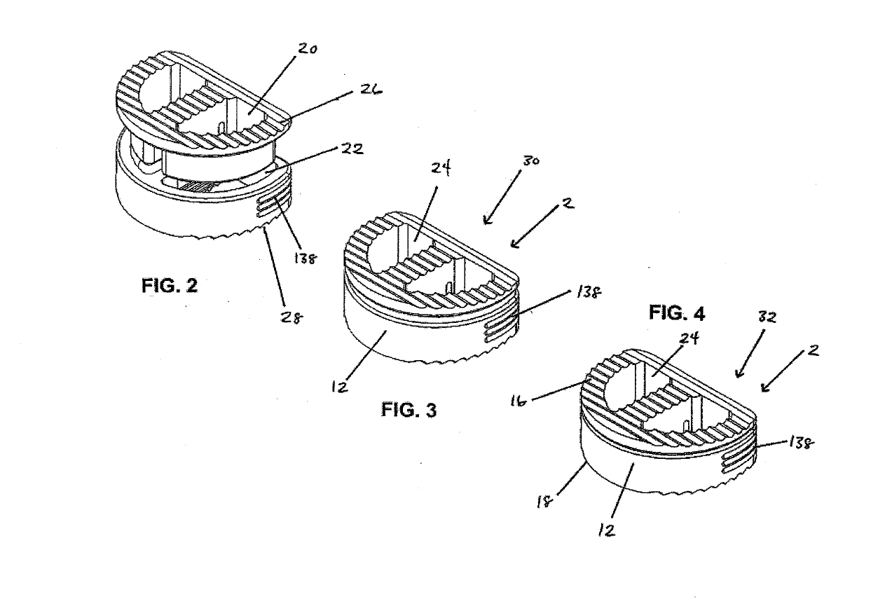

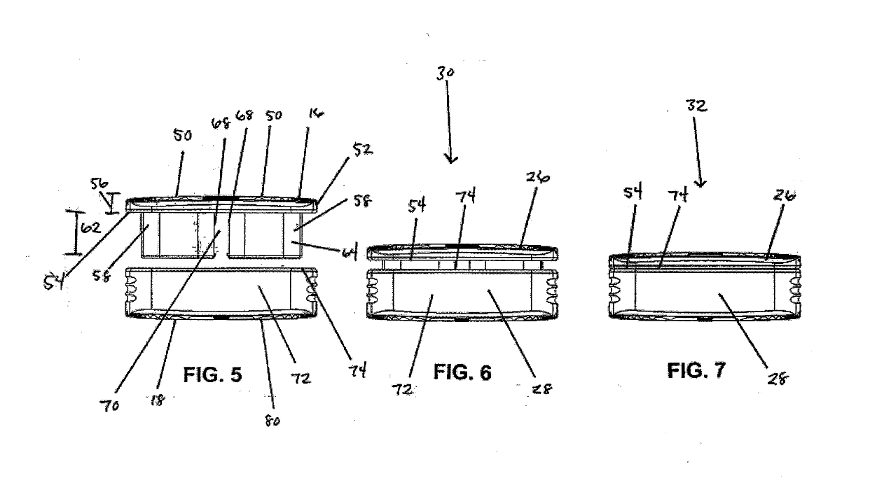

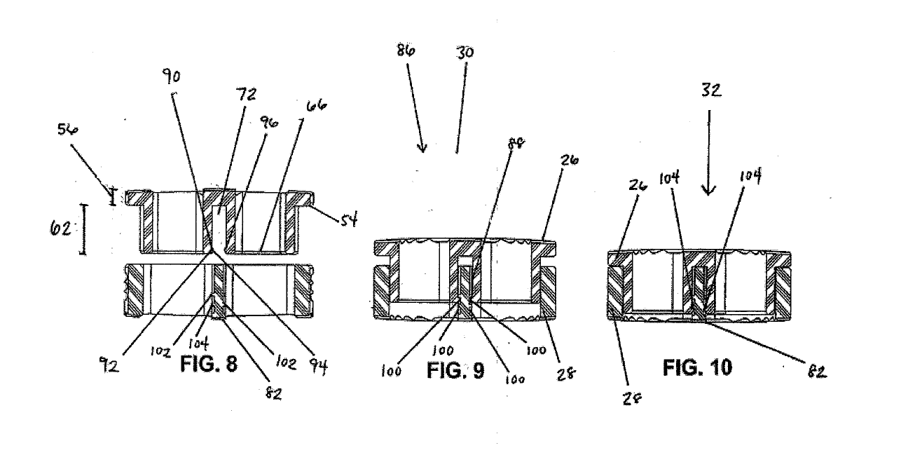

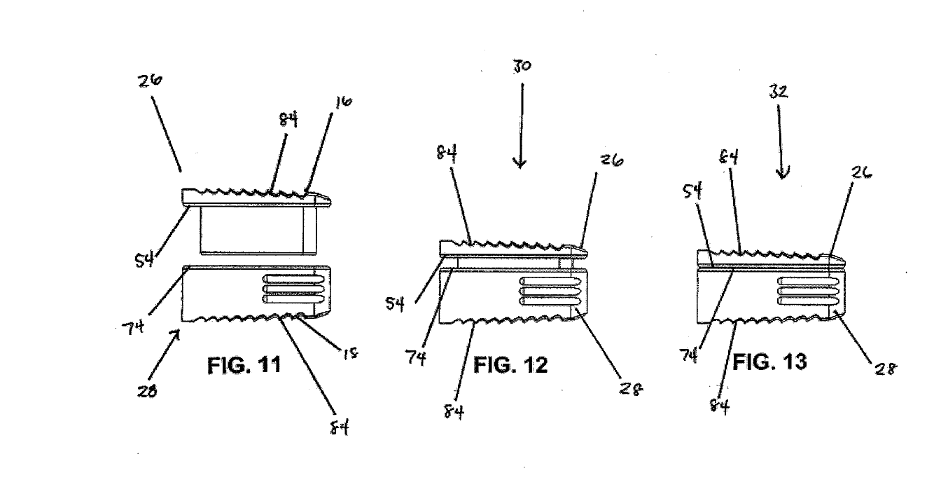

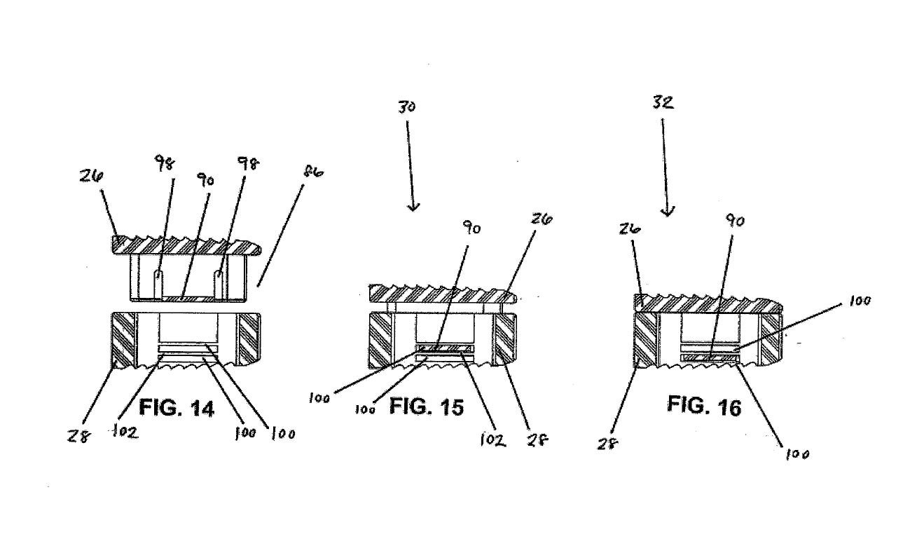

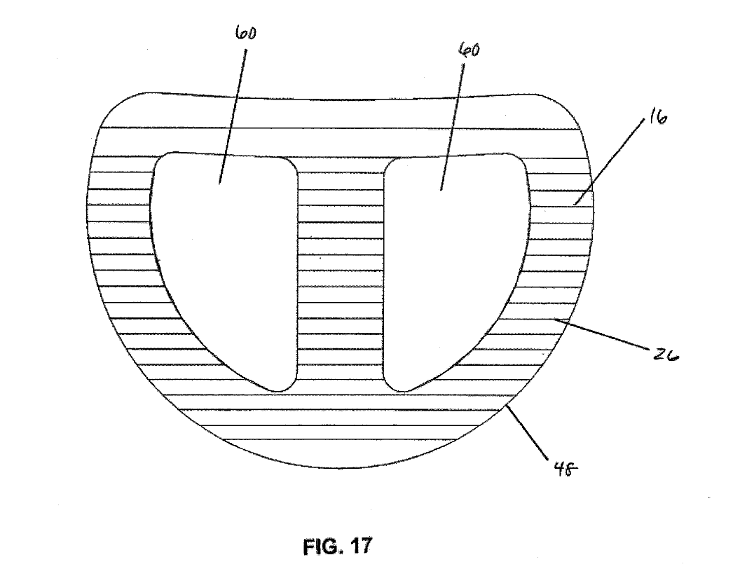

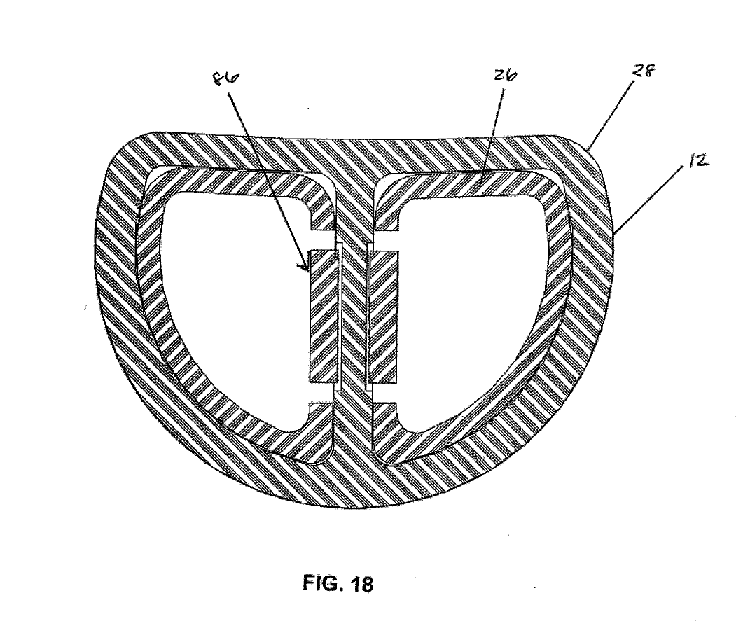

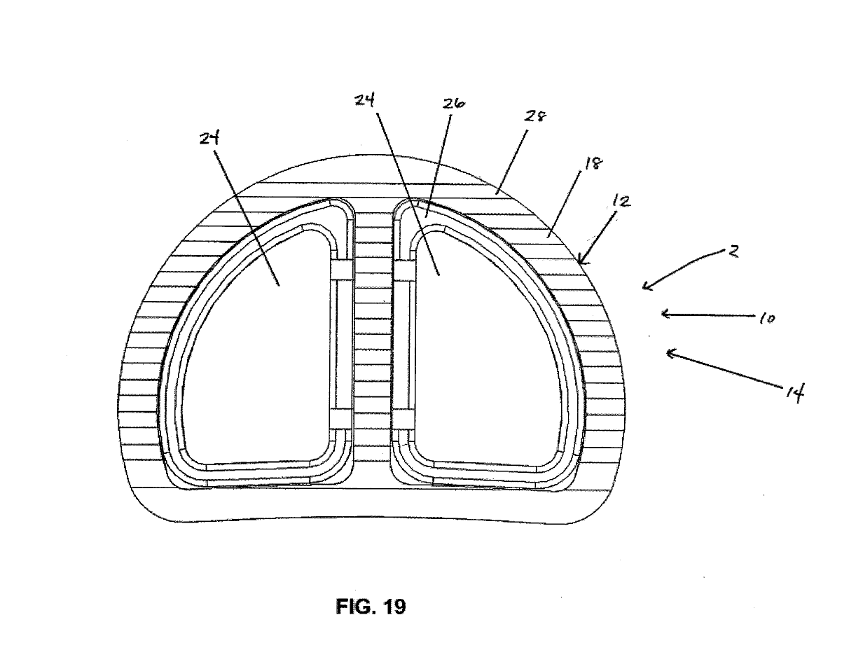

In FIGS. 1-19, intervertebral implants 2 are shown for immobilizing portions of the spine 4 and fusing adjacent vertebral bodies 6 and 8. The intervertebral implants 2 include an implant body 10 having upstanding outer wall portions 12 defining the footprint 14 of the implant 2, upper and lower vertebral body engaging surfaces 16 and 18, upper and lower openings 20 and 22 of the upper and lower vertebral body engaging surfaces 16 and 18, and a cavity 24 extending between the upper and lower openings 20 and 22. The intervertebral implant body 10 further includes a first implant extrusion member 26 and a second implant extrusion member 28 shiftable relative to one another and operable to change the volume of the implant cavity 24 from an enlarged pre-implantation configuration 30 to a smaller implantation configuration 32.

The implant cavity 24 is configured to receive a bone growth or graft material 34 therein. The implant cavity 24 is generally loaded or packed with bone graft material 34 in the enlarged pre-implantation configuration 30 so as to provide additional space for bone graft material 34 to be received. After the implant body 10 is placed between adjacent intervertebral bodies 6 and 8 of the spine 4 the first and second implant extrusion members 26 and 28 shift relative to one another such that the implant cavity 24 shifts to the smaller implantation configuration 32. As a result of the decreased implant cavity volume, a portion of the bone graft material 34 is extruded from the implant cavity 24 out of the upper and lower openings 20 and 22 of the implant body 10 thereby filling any gaps 36 between the bone graft material 34 and the vertebral endplates 38. In addition, bone graft material 34 can further fill gaps 36 between the upper and lower surfaces 16 and 18 of the implant body 10 and the vertebral endplates 38 thereby increasing the interface therebetween.

The bone graft material 34 can be autograft, allograft or artificial material. However, in order for the bone graft material 34 to be extrudable from out of the cavity 24, any bone material must be comminuted and combined with other materials, such a blood or bone marrow to provide a workable bone graft material 34. In particular, the bone graft material 34 should have a consistency such that the bone graft material 34 will retain its shape within the implant cavity 24 in the pre-implantation configuration 30 during implantation, and is extrudable from out of the cavity 24 and into gaps 36 between the bone graft material 34, upper and lower implant body surfaces 16 and 18 and the vertebral endplates 38. An exemplary bone graft material 34 consistency includes paste and putty.

Upon packing the implant cavity 24, sufficient bone graft material 34 is used to ensure that gaps 36 between the concave surface 40 of the vertebral endplate 38 and bone graft material 34 will be minimized or eliminated after implantation. Generally, the decrease in volume of the implant cavity 24 between the enlarged pre-implantation configuration 30 and the implantation configuration 32 should be sufficient such that the bone graft material 34 need not be packed above the upper and lower surfaces 16 and 18 of the implant body 10 while in the pre-implantation configuration 30. As such, shearing of bone graft material 34 during insertion is minimized or eliminated.

The implant 2 is configured to have a footprint 14 which is preferably less than the surface area 42 of the endplates 38 of the vertebrae between which the implant 2 is implanted. Exemplary dimensions include 30 mm by 23 mm, 36 mm by 27 mm, and 40 mm by 29 mm. Further, the implant height, in the implantation configuration 32, is configured to provide the appropriate spacing between adjacent vertebrae, such as from about 12 mm to about 20 mm.

Additionally, the upper and lower surfaces 16 and 18 of the implant 2 are configured to conform to the curvature of the endplates 38 of the vertebrae 6 and 8 so as to minimize gaps 36 therebetween.

In addition, the upper vertebral engaging surface 16 of the first implant extrusion member 26 and the lower vertebral engaging surface 18 of the second implant extrusion member 28 can include gripping structure 84 for engaging the faces of the vertebral bodies 6 and 8. The gripping structure 84 can include known gripping structure and configurations, such as individual teeth, ridges. Further, the gripping structure 84 can be configured to guide insertion of the implant 2 between adjacent vertebrae 6 and 8 and resist expulsion of the implant 2, such as with an angled gripping structure which allows the vertebral body 6 and 8 to generally slide therealong in one direction, while engaging and resisting shifting of the implant 2 in relation to the vertebral body in another direction.

Further, the implant 2 can include radio-opaque markers 44, such as pins, formed of any suitable radio-opaque material, such as tantalum. The markers 44 are configured to allow a surgeon to use radiographic equipment, such as x-ray, to determine the approximate location and orientation of the implant 2 within the intervertebral space 46. By viewing the implant markers 44, the relative orientation of the implant 2 can be determined and adjusted as desired. The radio-opaque markers 44 are described in more detail in U.S. Utility application Ser. No. 12/016,684, which is incorporated herein in its entirety.

In a first embodiment, as shown in FIGS. 1-19, the implant 2 includes a first implant extrusion member 26 and a second implant extrusion member 28. The first implant extrusion member 26 includes an upper portion 48 for engaging a vertebral body. The upper portion includes an upper vertebral engaging surface 16 for engaging a facing vertebral body 6, an opening 50 of the upper surface 16 and an annular outer edge 52. Further, the upper portion 48 includes a lower surface 54 for engaging the second implant extrusion member. In addition, the first implant extrusion member 26 includes a depending wall portion 58 extending about a first cavity portion 60. The depending portion 58 is offset from the outer edge 52 of the upper portion 48 and extends from the opening 50 of the upper portion 48 downwardly a predetermined distance 62. As shown in FIG. 1, the depending portion 58 includes a generally continuous arcuate wall portion 64 extending around the first cavity portion 60. The cavity 60 extends from the upper surface opening 50, through the upper portion 48 and the lower portion 58 to a lower portion opening 66.

As shown in FIGS. 1-4, the upper surface 16 of the first implant extrusion member 26 includes a pair of openings 50 therein, although more than two openings 50 are contemplated. The upper surface 16 includes a central portion 68 thereof extending between the pair of openings 50. The depending portion 58 includes a pair of depending portions 58 extending downwardly from each of the upper surface openings 50, including central wall portions 68 of each of the depending portions 58 facing one another cooperating to form a central sleeve portion 70.

The second implant extrusion member 28 includes an upstanding continuous arcuate outer wall portion 72 extending about a second cavity 76. The wall portion includes an upper surface 74 thereof for engaging and supporting the lower surface 54 of the upper portion 48 of the first implant extrusion member 26 and a lower surface 18 for engaging the endplate of a vertebral body 6 and 8, and a cavity 76. The second cavity 76 is configured to be packed with bone graft material 34 and receive the depending portion 58 of the first implant extrusion member 26 therein. The cavity 76 further includes an upper opening 78 of the upper surface 74 for receiving the depending portion 58 and a lower opening 80 of the lower surface 18 for bone graft material 34 to be extruded therefrom. As shown in FIG. 4, the upstanding outer wall 72 of the second implant extrusion member 28 extends a distance greater than the depending portion 58 of the first implant extrusion member 26 such that, when the upper surface 74 of the second implant extrusion member 28 engages the first implant extrusion member 26, the depending portion 58 does not extending beyond the lower surface 18 of the second implant extrusion member 28.

When the first extrusion member 26 includes spaced depending portions 58, such as shown in FIGS. 1 and 8-10, the second extrusion member 28 can include a central wall portion 82 extending across the second cavity 76, thereby defining a pair of second cavities 76. The central sleeve member 70 of the first extrusion member 26 is configured to fit over and receive the central wall portion 82 of the second implant extrusion member 28 therein.

The first and second implant extrusion members 26 and 28 include a pre-implantation configuration 30 wherein the depending portion 58 of the first implant extrusion member 26 is received by and extends partially into the cavity 76 of the second implant extrusion member 28. An implantation configuration 32 of the first and second implant extrusion members 26 and 28 is defined by the depending portion 58 shifted further into the cavity 76 of the second implant extrusion member 28 such that the lower surface 54 of the first implant extrusion member 26 engages the upper surface 74 of the second implant extrusion member 28.

The implant cavity 24 refers to the available volume between the cavities 60 and 76 of the first and second implant extrusion members 26 and 28. While the volume of the first implant extrusion member cavity 60 remains constant, the available volume of the second implant extrusion member cavity 76 varies with the positioning of the depending portion 58 of the first implant extrusion member 26 in the second cavity 76. More particularly, the available volume in the second cavity 76 is determined by the cavity walls 72, the lower surface 18 of the second implant extrusion member 28, and the depending portion 58 of the first implant extrusion member 26. Therefore, in the pre-implantation configuration 30, wherein the depending portion 58 is only partially received in the second cavity 76, the available volume is enlarged compared to the implantation configuration 32, wherein the depending portion 58 is fully received in the second cavity 76.

Prior to implantation, the first and second implant extrusion members 26 and 28 are positioned in the pre-implantation configuration 30 and the implant cavity 24 is packed full with bone graft material 34. After the implant 2 has been positioned between adjacent vertebrae 6 and 8, the distracted vertebrae 6 and 8 have the distraction tools removed from therebetween and are allowed to engage the upper and lower vertebral engaging surfaces 16 and 18. As a result of the compression by the vertebral bodies, the first and second implant members 26 and 28 shift to the implantation configuration 32. As the depending portion 58 of the first implant extrusion member 26 shifts further into the cavity 76 of the second implant extrusion member, the implant cavity volume decreases. An amount of bone graft material 34 corresponding to the decreased cavity volume is extruded from the upper opening 50 of the first extrusion member 26 and the lower opening 80 of the second extrusion member 28. As discussed above, both of the implant extrusion members 26 and 28 include wall portions 64 and 72 defining a portion of the implant cavity 24, the wall portions 64 and 72 cooperating to resist bone graft material 34 extrusion from anywhere but the upper and lower openings 50 and 80 of the implant extrusion members 26 and 28. Note that a negligible amount of the bone graft material 34 may enter between the sleeve portion 70 of the first extrusion member 26 and the central wall portion 82 of the second extrusion member 28 upon shifting the implant extrusion members 26 and 28 to the implantation configuration 32.

The extruded bone graft material 34 fills any gaps 36 or voids between it and the concave endplates 6 and 8 and further can fill gaps 36 between the upper surface 16 of the first implant extrusion member 26 and the adjacent vertebral face 6 and 8 and between the lower surface 18 of the second implant extrusion member 28 and the adjacent vertebral face 6 and 8. As a result, the vertebral endplates 6 and 8 are engaged by implant 2 and the bone graft material 34.

As discussed above, the implantation configuration is defined by the depending portions 58 of the first implant extrusion member 26 being fully received in the cavity 76 of the second implant extrusion member 28. The pre-implantation configuration 30 of the implant extrusion members 26 and 28 is defined by the depending portions 58 being partially received in the cavity 76 of the second implant extrusion member 28. Preferably, a mechanical connection 86 biases the extrusion implant members 26 and 28 in the pre-implantation configuration 30 to allow for insertion of the implant 2 between adjacent vertebrae 6 and 8, while also allowing the implant extrusion members 26 and 28 to shift to the implantation configuration 32 upon the application of spinal loading to the upper and lower vertebral engaging surfaces 16 and 18 of the implant extrusion members 26 and 28.

Exemplary mechanical connections 86 include harpoons, spikes, or directionally positioned teeth of the sleeve portion 70 or central wall portion 82. Penetration of the spike, harpoon, or teeth would resist disengagement or decompression of the first and second implant extrusion members 26 and 28.

As shown in FIGS. 1, 8-10 and 14-16, the implant extrusion members 26 and 28 have a ratchet connection 88 to allow the implant extrusion members 26 and 28 to shift from the pre-implantation configuration 30 to the implantation configuration 32 and resist travel of the implant extrusion members 26 and 28 back to the pre-implantation configuration 30. As shown, the sleeve portion 70 of the first implant extrusion member 26 includes a boss portion 90 extending normally therefrom along the lower surface 92 thereof. The boss portion 90 includes a chamfered lower surface 94 and a flat upper surface 96. Further, extending vertically along either side of the boss portion 90 are a pair of slots 98 for allowing the boss portion 90 to be shifted from the sleeve portion into the first cavity 60 of the first implant extrusion member 26 as the chamfered surface 94 of the boss portion 90 is engaged.

To accommodate the boss portion 90, the central wall portion 82 of the second implant extrusion member 28 includes spaced recesses 100 therein to receive the boss portion 90, the recesses defining both the pre-implantation and implantation configurations 30 and 32. Upon the application of force to the upper and lower vertebral engaging surfaces 16 and 18 of the first and second implant extrusion members 26 and 28, the chamfered surface 94 of the boss portion 90 engages a non-recessed portion 102 of the central wall portion 82 and is urged into the first cavity portion 60 until the boss portion 90 encounters a recessed portion 100, whereat the boss portion 90 shifts into the recessed portion 100. Retraction of the first implant extrusion member 26 is resisted by the engagement of the flat upper surface 96 of the boss portion 90 and an upper flat step surface 104 of the recessed portions 100.

In an alternative form, the mechanical connection 86 is configured to allow for implant height variability upon varying spinal loading. The mechanical connection 86 can include a resistance mechanism 108 that is at rest in the pre-implantation configuration 30, and while allowing the implant extrusion members 110 and 112 to be shifted to the implantation configuration 32, will continue to urge the implant extrusion members 110 and 112 toward the pre-implantation configuration 30, such as a spring. Upon any given loading, the resistance mechanism 108 will allow the extrusion members 110 and 112 to shift to a given relationship wherein the resistive force of the resistance mechanism 108 is equilibrated with the spinal loading subjected to the implant 114. As the spinal loading decreases, the implant extrusion members 110 and 112 will shift toward the pre-implantation configuration 30. As the spinal loading increases, the implant extrusion members 110 and 112 will shift away from the pre-implantation configuration 30 toward a fully compressed configuration 32.

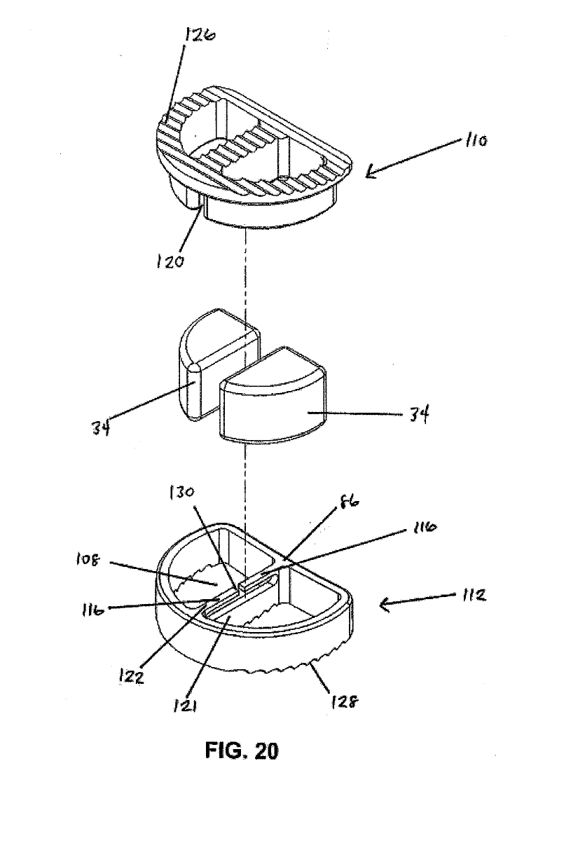

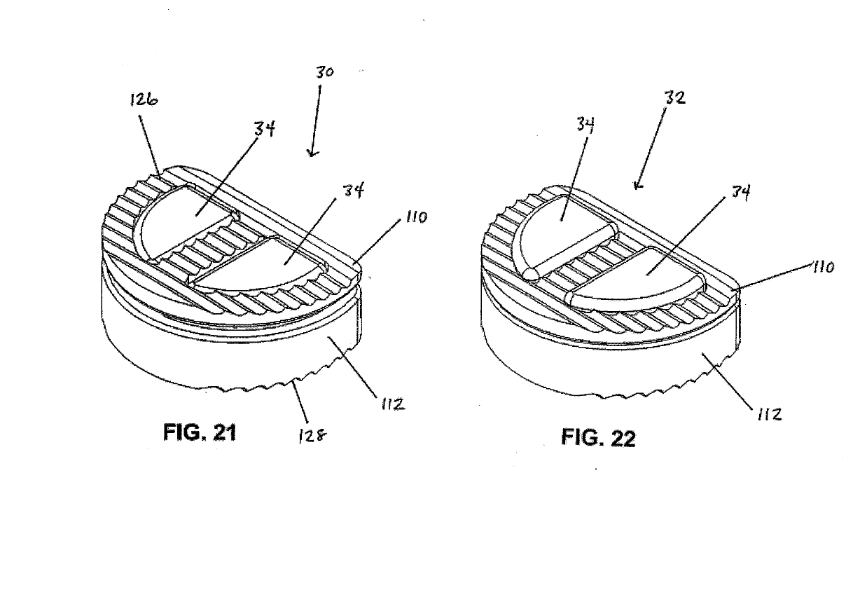

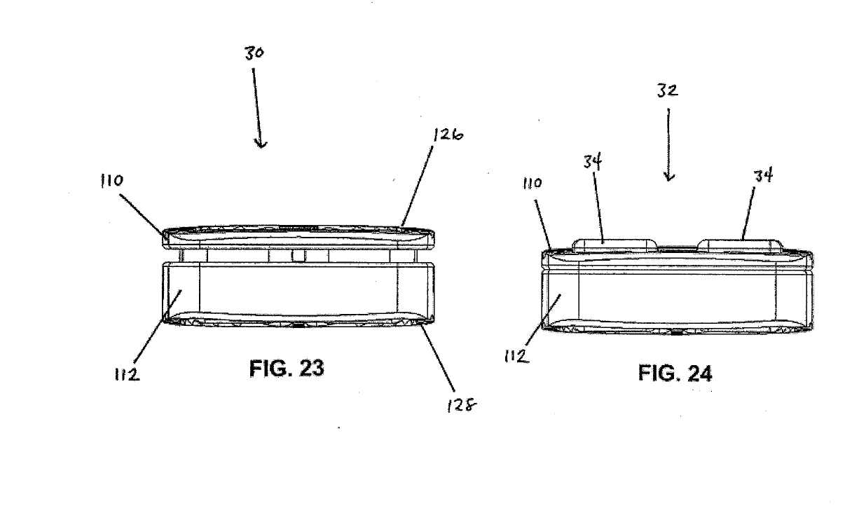

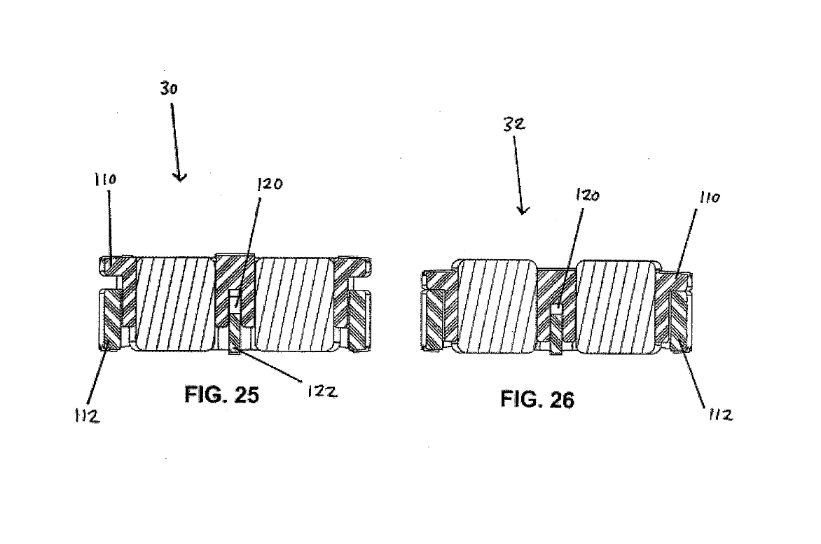

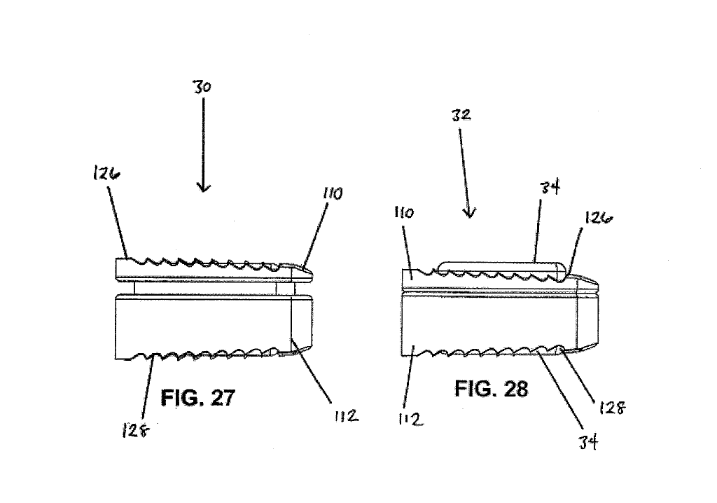

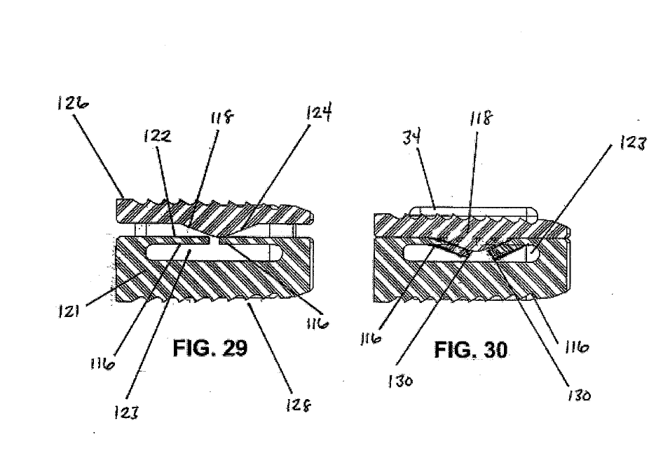

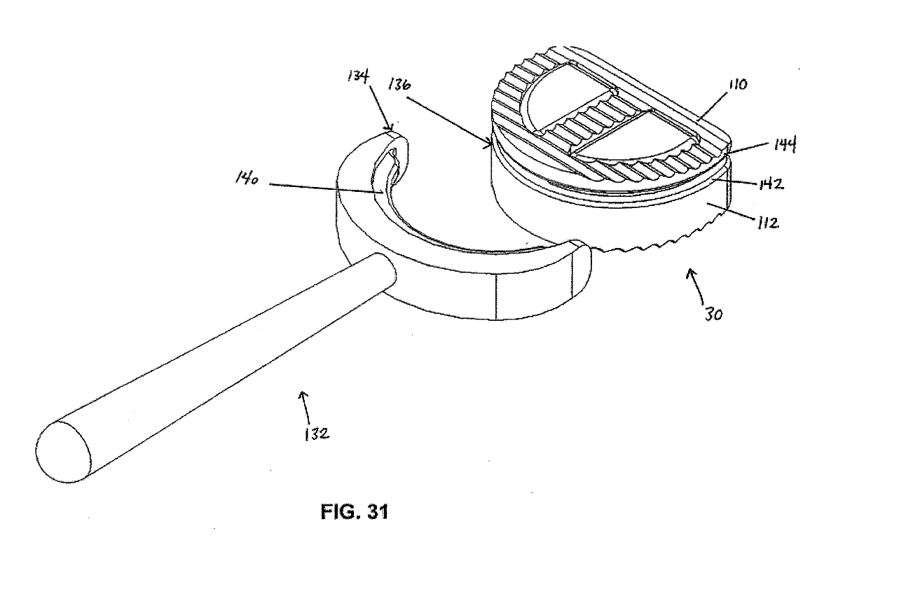

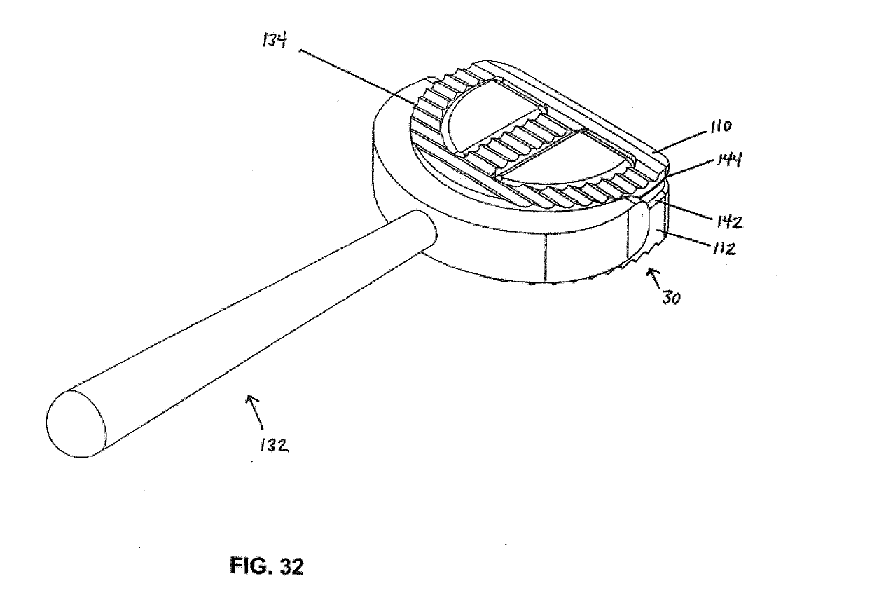

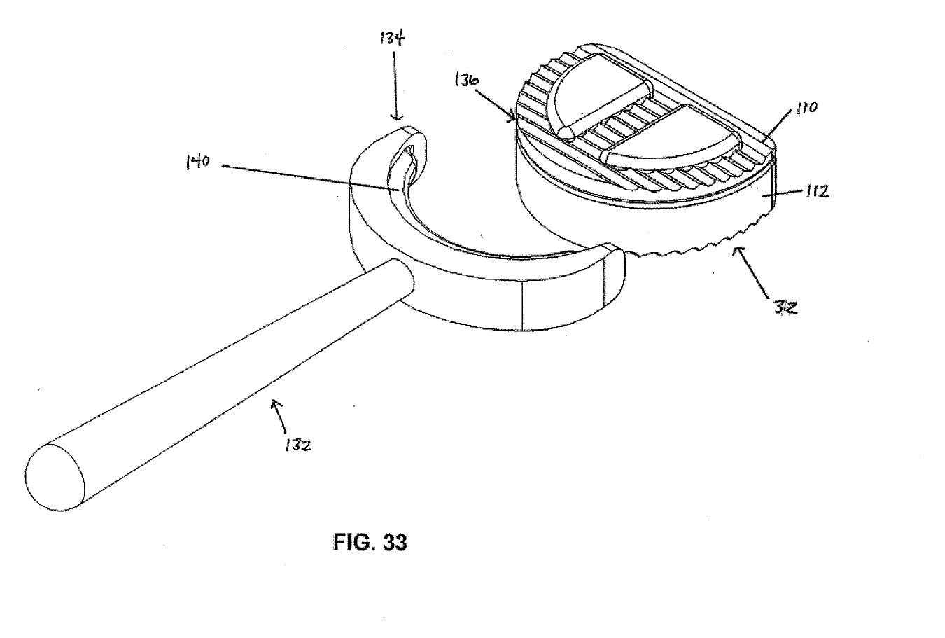

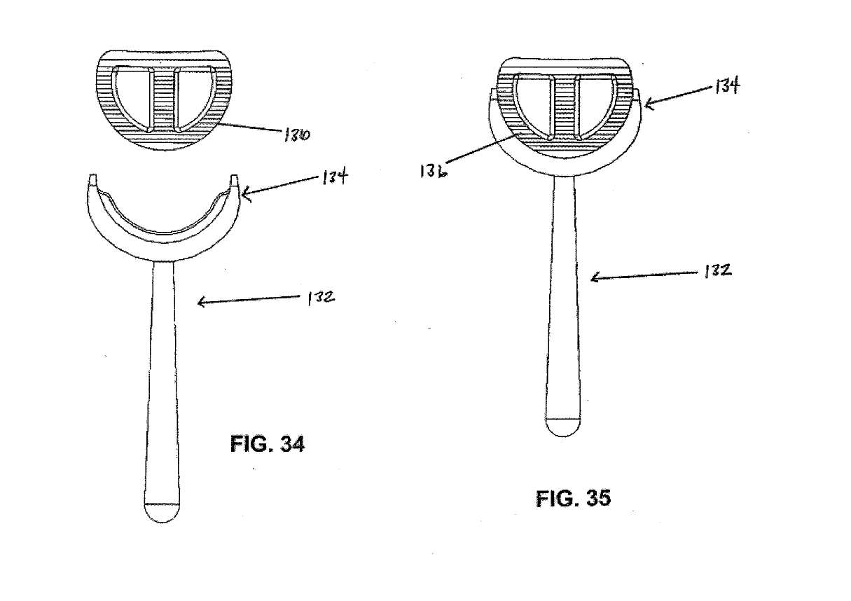

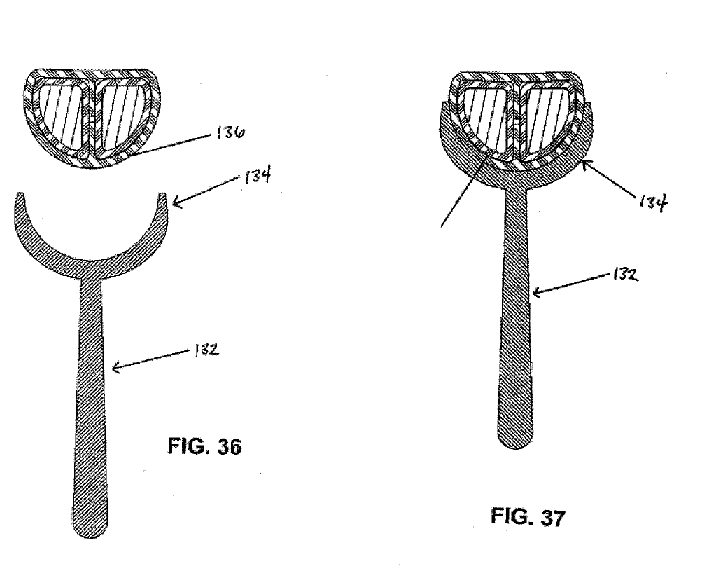

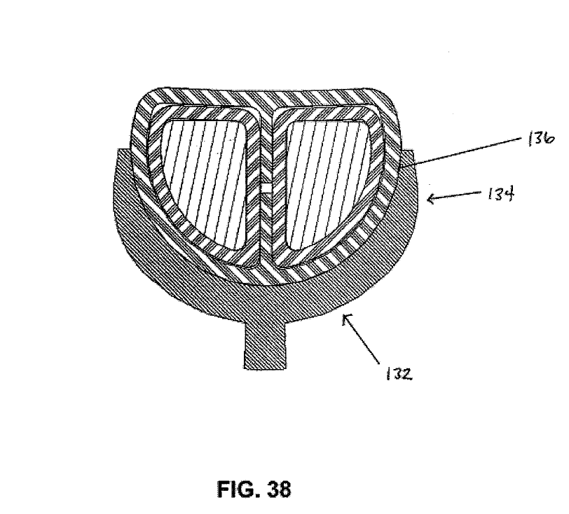

As shown in FIGS. 20-38, the resistance mechanism 108 of the implant extrusion members 110 and 112 includes cantilevered portions 116 of the second implant extrusion member 112 and a wedge portion 118 of the first implant extrusion member 110. As shown in FIGS. 29 and 30, the first implant extrusion member 110 includes a tapered wedge portion 118 extending downwardly in the sleeve portion 120. As further shown in FIGS. 29 and 30, the central wall portion 121 of the second implant extrusion member 112 includes a pair of spaced cantilevered portions 116 and a slot 123 below the spaced cantilevered portions 116 to allow the cantilevered portions 116 to freely deflect downwardly therein upon the application of force to the upper surfaces 122 of the cantilevered portions 116.

As shown in FIG. 29, in the pre-implantation configuration 30 the lower surface 124 of the wedge 118 of the first implant extrusion member 110 engages the upper surface 122 of the cantilevered portions 116, resulting in downward deflection of the cantilevered portions 116. After the implant is positioned between adjacent vertebrae 6 and 8 and subjected to spinal loading, the upper and lower vertebral engaging surfaces 126 and 128 of the implant are engaged and urge the lower surface 124 of the wedge 118 and the upper surfaces 122 of the cantilevered portions 116 toward one another. As a result of the loading, the wedge 118 engages distal portions 130 of the cantilevered portions 116 thereby causing the distal end 130 to further deflect downwardly into the central wall slot 123, such as shown in FIG. 30. As spinal loading increases and decreases the amount of deflection of the cantilevered portions 116 will vary.

As shown in FIGS. 31-38, an exemplary insertion tool 132 includes a crescent shaped implant engaging portion 134 configured to receive the wall portion 136 of the second extrusion member 112 therein. Further, the engaging portion 134 is configured to engage the implant without any holes or penetrations in the implant. As shown in FIGS. 1-4, the wall portion 136 of the second implant extrusion member 112 can include surface grooves 138 therein for receiving the tool 132.

Additionally, the crescent shaped engagement portion 134 further includes a flange 140 extending therealong for being received between the upper surface 142 of the second implant extrusion member 112 and the lower surface 144 of the first implant extrusion member 110. As a result, the tool 132 assists in maintaining the implant in the pre-implantation configuration 30 until the tool 132 is disengaged from the implant. More particularly, the flange 134 prevents the first and second implant extrusion members 110 and 112 from shifting prematurely to the implantation configuration 32.

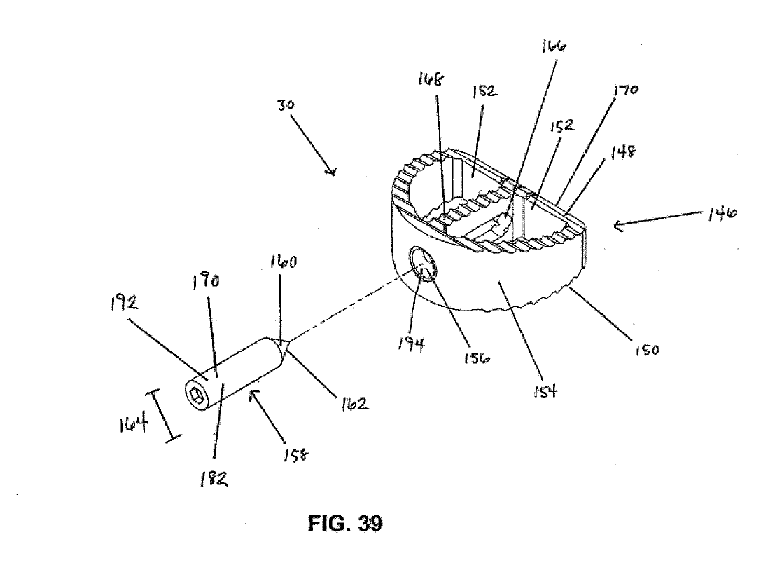

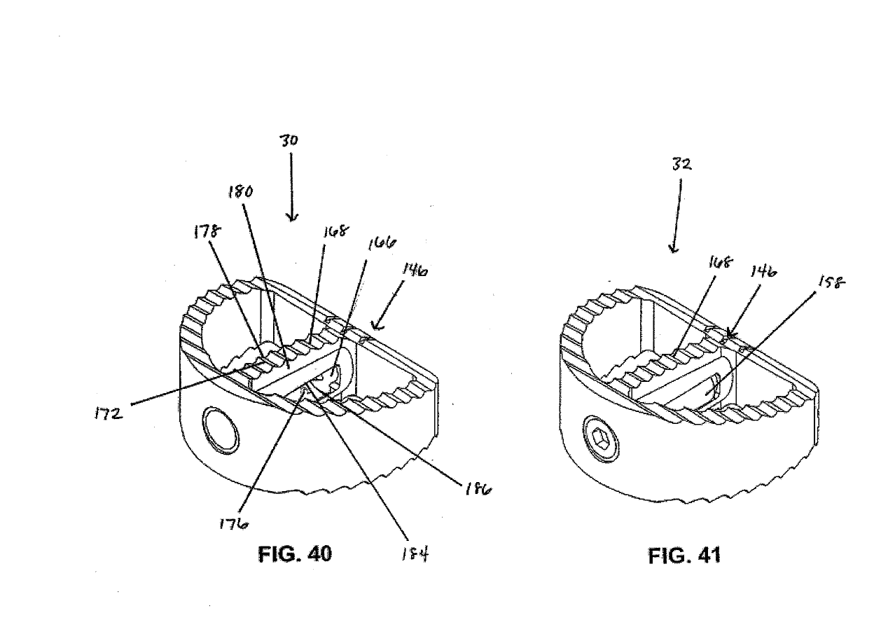

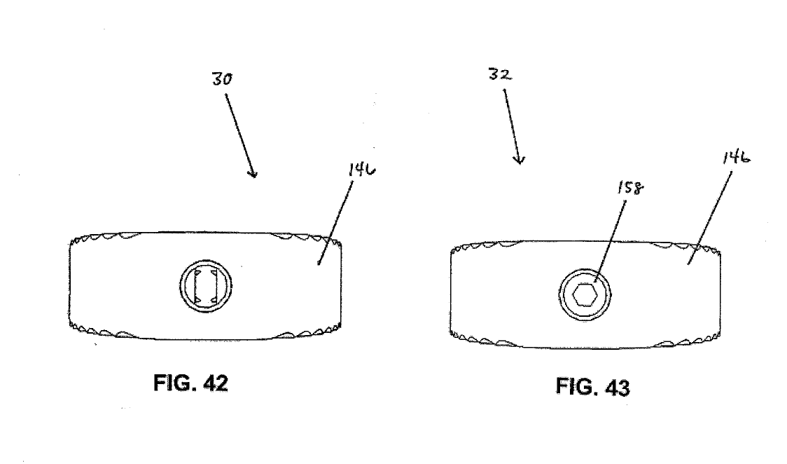

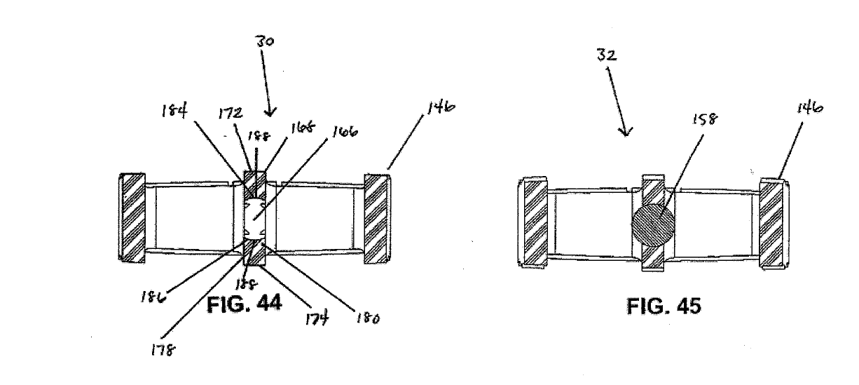

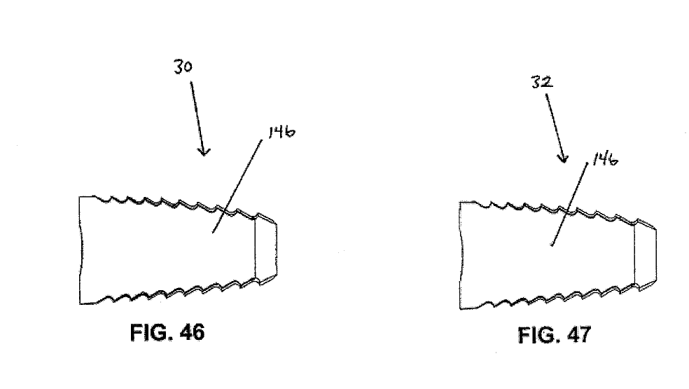

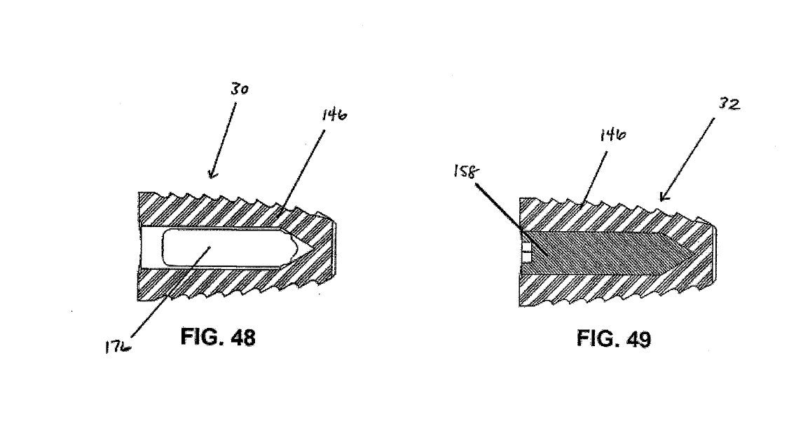

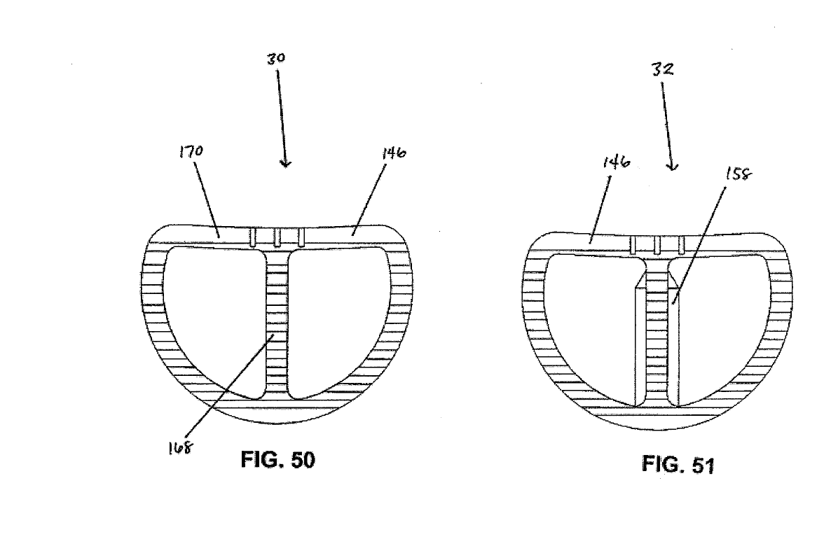

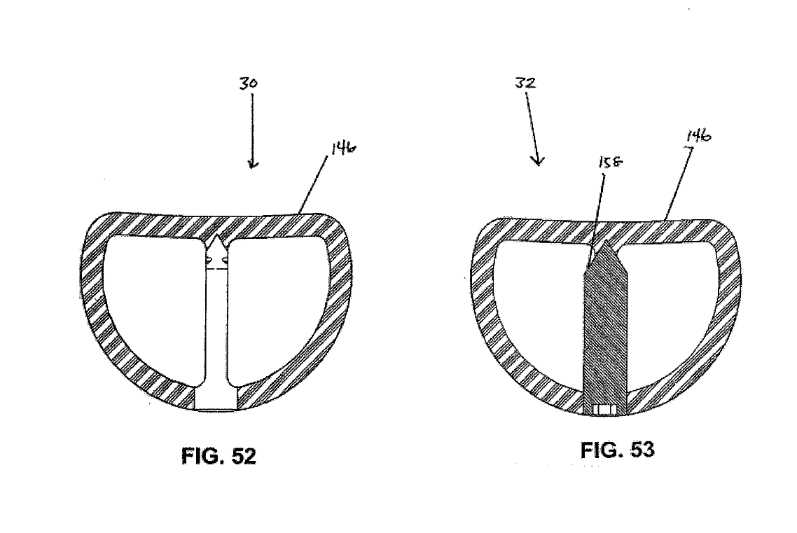

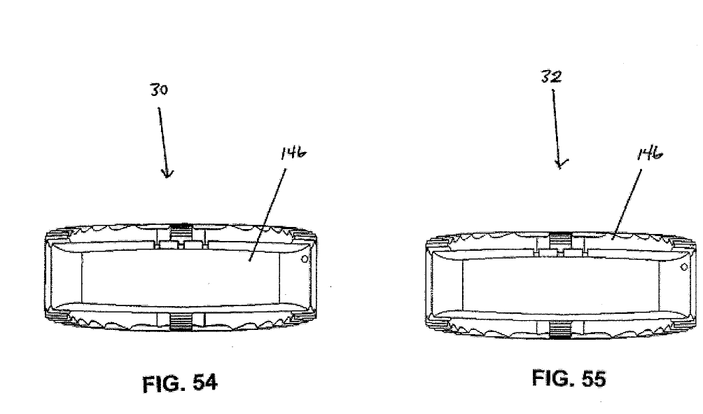

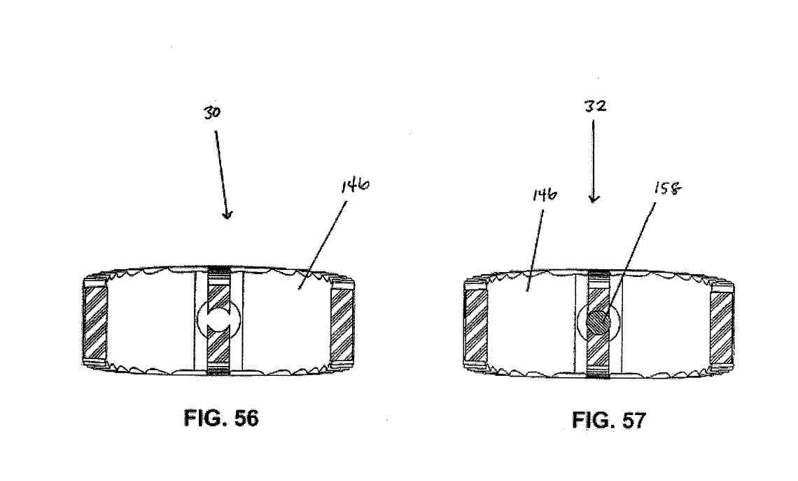

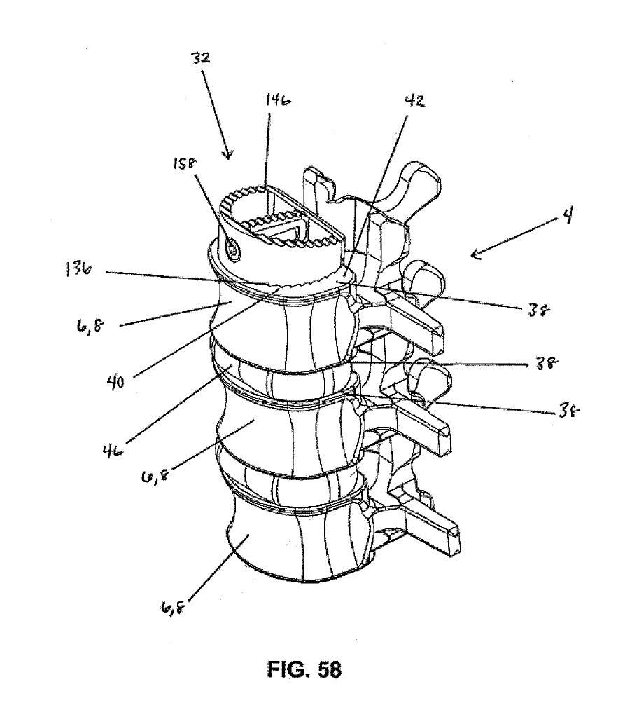

In a third embodiment, such as shown in FIGS. 39-58, the first implant extrusion member 146 includes an arcuate wall 154 extending about a cavity 152. The wall 154 further includes upper and lower vertebral engagement surfaces 148 and 150 for engaging adjacent vertebrae 6 and 8. The wall 154 of the first implant extrusion member 146 further includes a throughbore 156 for receiving the second implant extrusion member 158 therein and allowing the second implant extrusion member 158 to shift therethrough to the implantation configuration 32.

To ease insertion of the second implant extrusion member 158 into the cavity 152 and through the bone graft material 34, the distal end 160 thereof can be tapered, such as with a conical distal end configuration 162 as shown in FIG. 39.

The pre-implantation configuration 30 of the first and second implant extrusion members 146 and 158 can be defined by the second implant extrusion member 158 being at any location other than that defining the implantation configuration 32. As such, the pre-implantation configuration 30 can include the second implant extrusion member 158 extending partially into cavity 152, being received in the throughbore 156 but not extending into the cavity 152, or not being received in the throughbore 156 of the first implant extrusion member 146. For any given procedure, the exact location of the second implant extrusion member 158 can be adjusted to achieve the desired amount of bone graft material 34 extrusion.

Additionally, the amount of bone graft material 34 extruded from the cavity 152 can be controlled by the distance the second implant extrusion member 158 shifts into the cavity 152.

Preferably, the distal end 160 of the second implant extrusion member 158 is supported in the implantation configuration 32 by the first implant extrusion member 146 so as to minimize shifting of the second implant extrusion member 158 during the application of spinal loading on the implant and bone graft material 34. An exemplary support can include a support portion 166 of the wall opposite the throughbore 156 of the first implant extrusion member 146 configured to receive and support the distal end 160 of the second implantation extrusion member 158 therein.

Another support, as shown in FIGS. 39-41, includes a central wall 168 of the first implant extrusion member 146 extending from the arcuate wall 154 having the throughbore 156 therein to an opposite wall 170 of the first implant extrusion 146 member. As shown in FIG. 41, the upper and lower surfaces 172 and 174 of the central wall 168 can correspond to the upper and lower surfaces 148 and 150 of the arcuate outer wall 154 of the first implant extrusion member 146.

The central wall 168 includes a slot 176 therein extending from the wall throughbore 156 and toward the opposite wall 170. The slot 176 is configured to be packed with bone graft material 34 and receive the second implant extrusion member 158 therein as the second implant extrusion member 158 shifts toward the implantation configuration 32. However, as shown in FIGS. 45 and 51, the slot 176 need not necessarily be packed with bone graft material 34 in the pre-implantation configuration 30 as the second implant extrusion member 158 can be configured to extend beyond the side surfaces 178 and 180 of the central wall portion 168, thereby displacing bone graft material 34 within the cavity 152 but not necessarily in the slot 176 as the second implant extrusion member 158 shifts to the implantation configuration 32.

As shown in FIGS. 39, the second implant extrusion member 158 can include an annular outer surface 182 with the upper and lower slot defining surfaces 184 and 186 of the central wall portion 168 each having an arcuate configuration 188 corresponding to the annular outer surface 182 of the second implant extrusion member 158.

The engagement of the throughbore 156 and the outer surface 182 of the second implant extrusion member 158 are configured to minimize and preferably mitigate extrusion therebetween. One exemplary interface includes a threaded annular outer surface 192 of the second implant extrusion member 158 and a corresponding inner threaded annular surface 194 of the throughbore 156.

Alternatively, the first implant extrusion member 146 can include multiple wall portion throughbores 156. The throughbores 156 can be utilized to insert more than one second implant extrusion member 158 into the cavity 152, thereby increasing the volume of bone graft extrusion. Additionally, the throughbores 156 not intended to be occupied by second implant extrusion members 158 can be filled or patched to prevent extrusion of bone graft material 34 therethrough as the first and second implant extrusion members 146 and 158 shift to the implantation configuration 32.

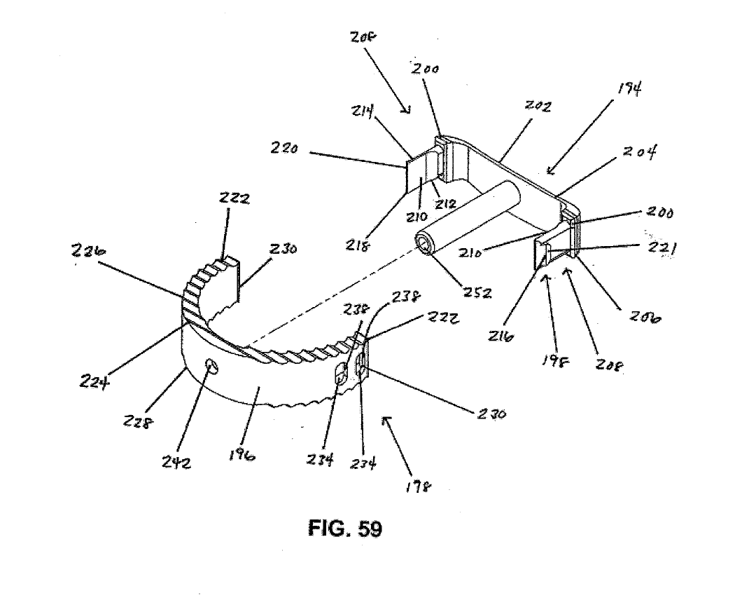

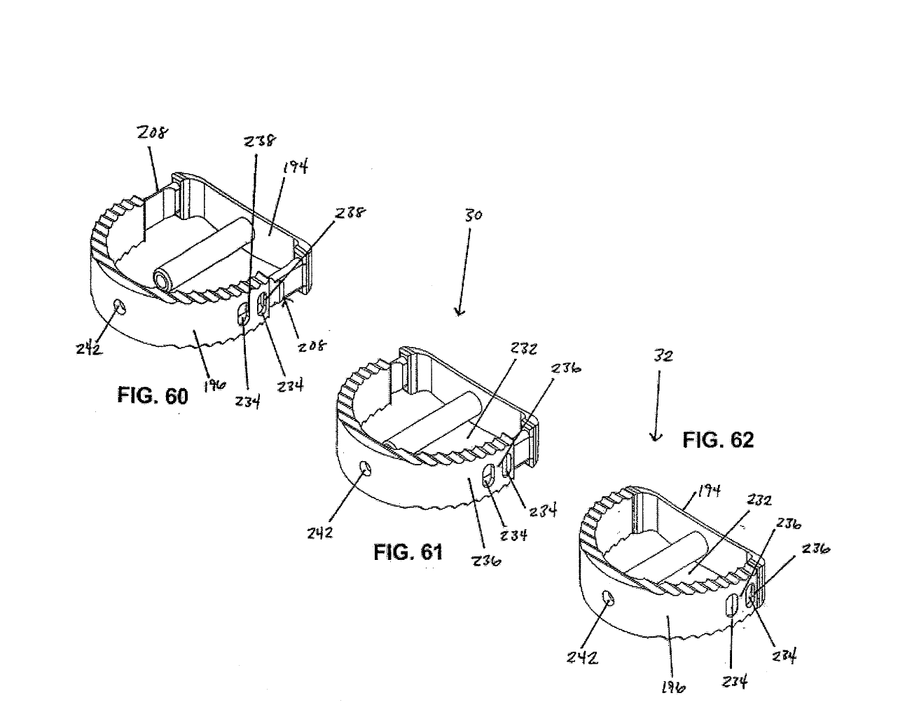

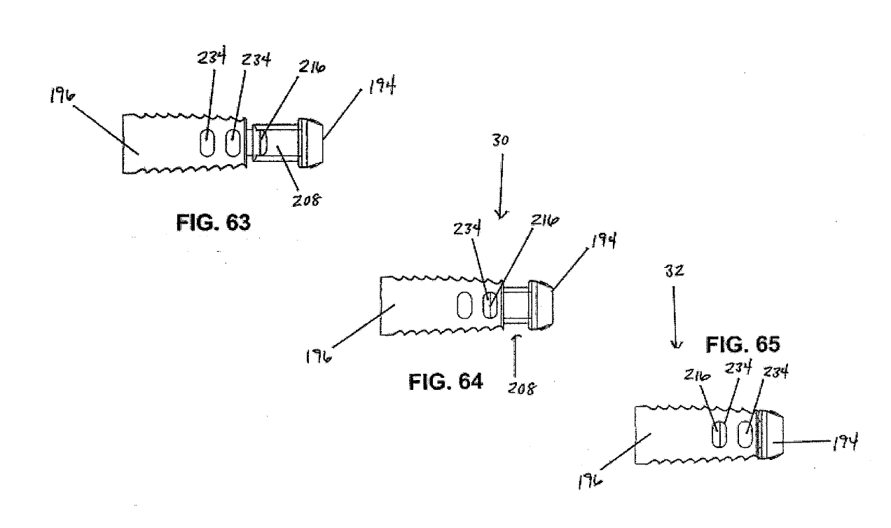

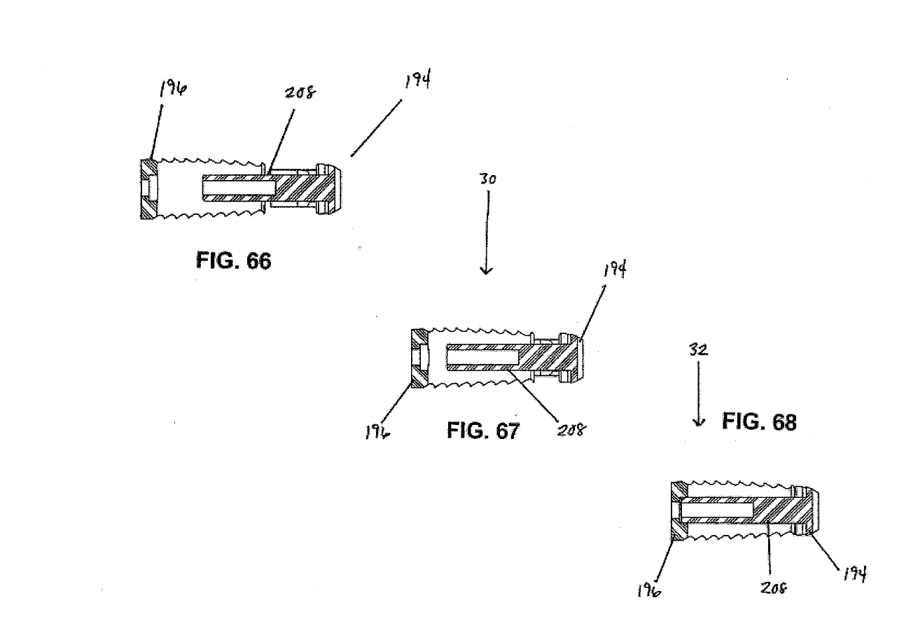

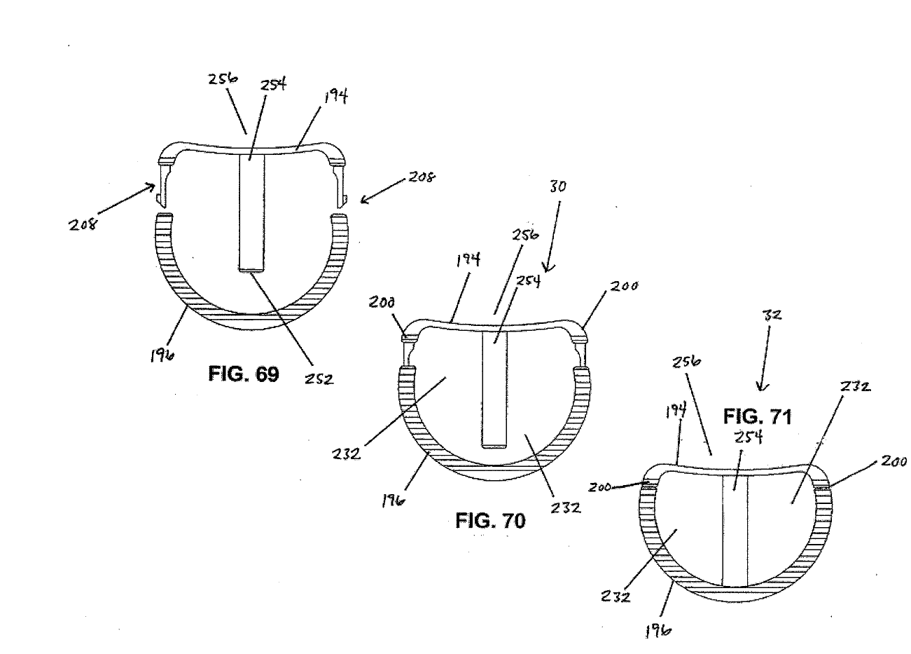

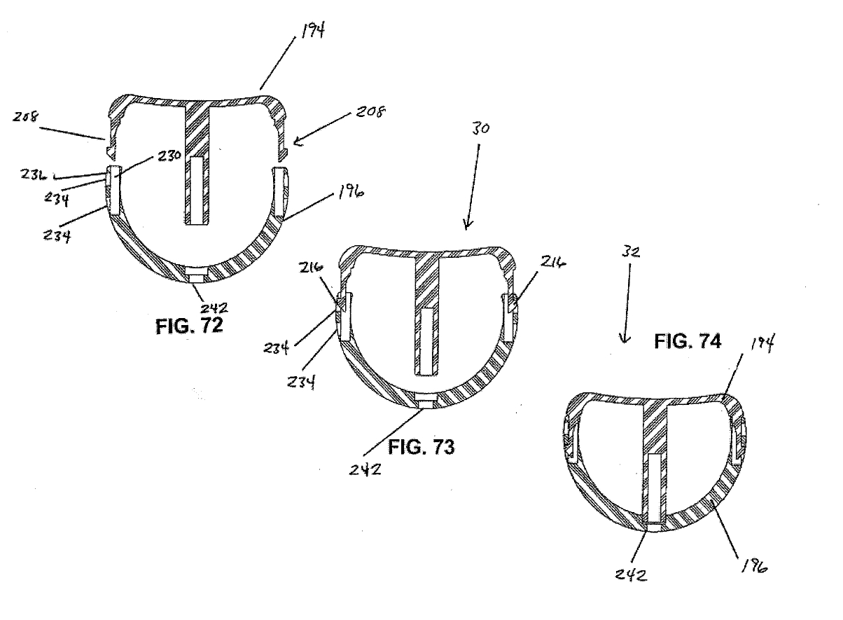

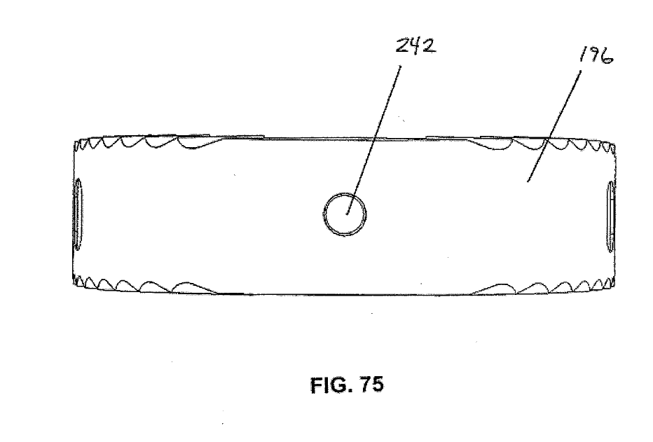

In a fourth embodiment, as shown in FIGS. 59-75, the first and second implant extrusion members 194 and 196 have a ratchet connection 198 and are configured to be shifted laterally along the ratchet connection 198 relative to one another between adjacent vertebrae 6 and 8 from the pre-implantation configuration 30 to the implantation configuration 32. As shown in FIGS. 59 and 60, the first implant extrusion member 194 includes an arcuate wall 202 having opposite ends 200 thereof. The arcuate wall portion 202 includes a first upper vertebral engaging surface 204 and a first lower vertebral engaging surface 206 for engaging the faces of adjacent vertebral bodies 6 and 8.

Further, the end portions 200 of the first implant extrusion member 194 include pawl portions 208 extending therefrom. The pawl portions 208 include a wall portion 210, with the lower surface 212 of the pawl wall portion 210 positioned above the first lower vertebral engaging surface 206 and the pawl wall upper surface 214 positioned below the first upper vertebral engaging surface 204. The pawl portion 208 further includes a wedge portion 216 attached to the distal end 218 thereof, with the wedge thickness 220 being the thinnest adjacent the pawl distal end 218. The wedge portion 216 further includes a flat back wall surface 221 extending generally perpendicular from the pawl wall 210.

The second implant extrusion member 196, as shown in FIGS. 59 and 60, includes an arcuate wall 224 and a pair of second end portions 222 at either end thereof. The second arcuate wall 224 includes a second upper vertebral engaging surface 226 and a second lower vertebral engaging surface 228 for engaging adjacent vertebrae. Further, the second end portions 222 include openings 230 therein for receiving the pawl portions 208 of the first extrusion member 194.

The first and second implant extrusion members 194 and 196 define a cavity 232 therebetween in the pre-implantation and implantation configurations 30 and 32. The pre-implantation configuration 30 as shown in FIG. 61 is defined by the pawl portions 208 of the first implant extrusion member 194 being partially received in the end openings 230 of the second implant extrusion member 196. The end openings 230 include recessed portions or openings 234 therein for receiving the pawl wedge portions 216 in both the pre-implantation and implantation configuration 30 and 32. As the pawl wedge portions 216 shift along the non-recessed portions 236 of the end openings 230, the pawls 208 are urged from their normal position. As the pawl wedge portions 216 encounter the recessed portions 234 of the second end openings 230, the pawls 208 are allowed to return to their normal orientations, thereby defining either the pre-implantation configuration 30 or the implantation configuration 32. In both configurations 30 and 32, a wall portion 238 of the recessed portion 234 is configured to engage the back wall portion 220 of the pawl wedge 216, restricting movement of the pawl portions 208 out of the second end openings 230.

In the pre-implantation configuration 30, the implant cavity 232 is defined by the walls 210 and 224 of the first and second implant extrusion members 194 and 196 and the pawl wall portions 210 not received in the second end openings 230. The implant cavity 232 can be packed with bone graft material 34 in the pre-implantation configuration 30. As the first and second implant extrusion members 194 and 196 are shifted toward one another toward the implantation configuration 32, the implant cavity 232 is defined only by the wall portions 202 and 224- of the first and second implant extrusion members 194 and 196.

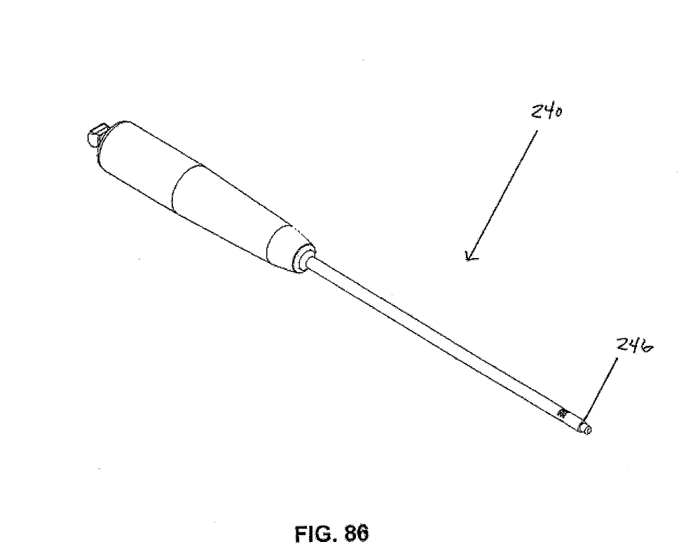

A tool 240 such as shown in FIG. 86 can be used to shift the first and second implant extrusion members 194 and 196 from the pre-implantation configuration 30 to the implantation configuration 32. As shown in FIGS. 59 and 72-74, the second implant extrusion member 194 can include a tool opening 242 in the wall portion 224. Further, the tool socket opening 242 is obstructed by the tool 240 such that bone graft material 34 packed in the cavity 232 while the first and second implant extrusion members 194 and 196 are shifted to the implantation configuration 32.

Further, the first implant extrusion member 194 can include a threaded opening 252 for receiving the threaded tool portion therein 246. As shown in FIGS. 59 and 69-74, the first implant extrusion member 194 includes a cantilever shaft portion 254 extending from a generally central area 256 between the first end portions 200 and into the implant cavity 232. The cantilever shaft portion 254 includes a threaded opening 258 at a distal end 260 thereof for receiving the threaded tool portion 246.

As a result, when the first and second implant extrusion members 194 and 196 are in the pre-implantation configuration 30, and the tool 240 is engaged in the socket opening 242 and the threaded tool portion 246 is engaged with the threaded shaft opening 252, the implant cavity 232 can be packed with bone graft material 34. After the first and second implant extrusion members 194 and 196 are positioned between the adjacent vertebrae 6 and 8, the threaded tool portion 246 can be used to urge the first and second implant extrusion members 194 and 196 toward one another and into the implantation configuration 32. After the implant extrusion members 194 and 196 are in the implantation configuration 32 and the bone graft material 34 has been extruded from the implant cavity 232, the threaded tool portion 246 of the tool 240 can be removed. The socket opening 242 can optionally be filled thereafter.

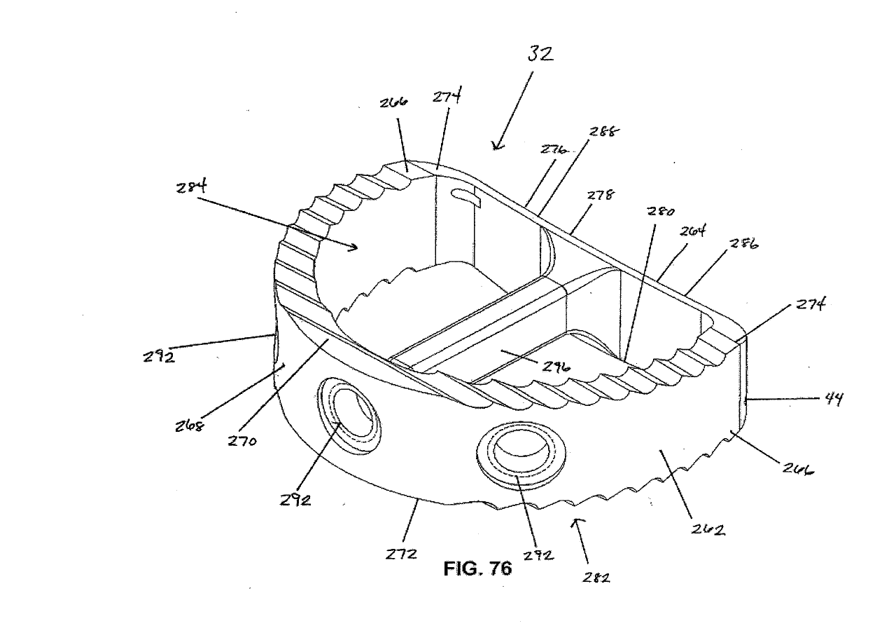

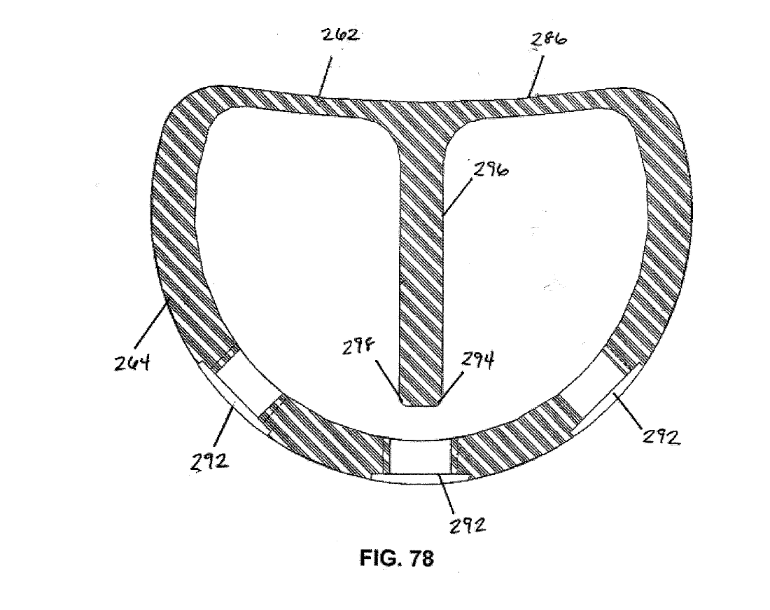

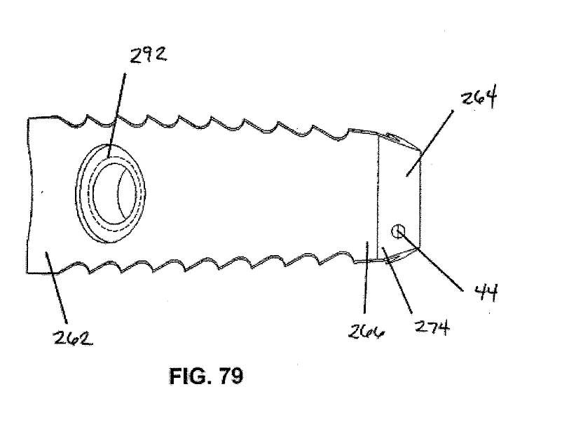

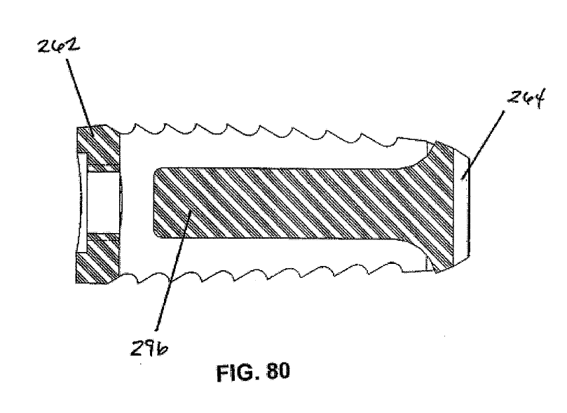

Alternatively, the first and second implant extrusion members 262 and 264 can be connected to one another with one or both of the implant extrusion members 262 and 264 being deformable or adjustable between a pre-implantation 30 and implantation configuration 32. As shown in FIGS. 76-80, in a fifth embodiment the first implant extrusion member 262 includes a rigid arcuate wall 268 having end portions 266 thereof. The rigid arcuate wall portion 268 includes a first upper vertebral engaging surface 270 and a lower vertebral engaging surface 272 for engaging the faces of adjacent vertebral bodies 6 and 8. The second implant extrusion member 264 includes a deformable wall 276 extending between a pair of second end portions 274. The deformable wall 276 includes a second upper vertebral engaging surface 278 and a second lower vertebral engaging surface 280.

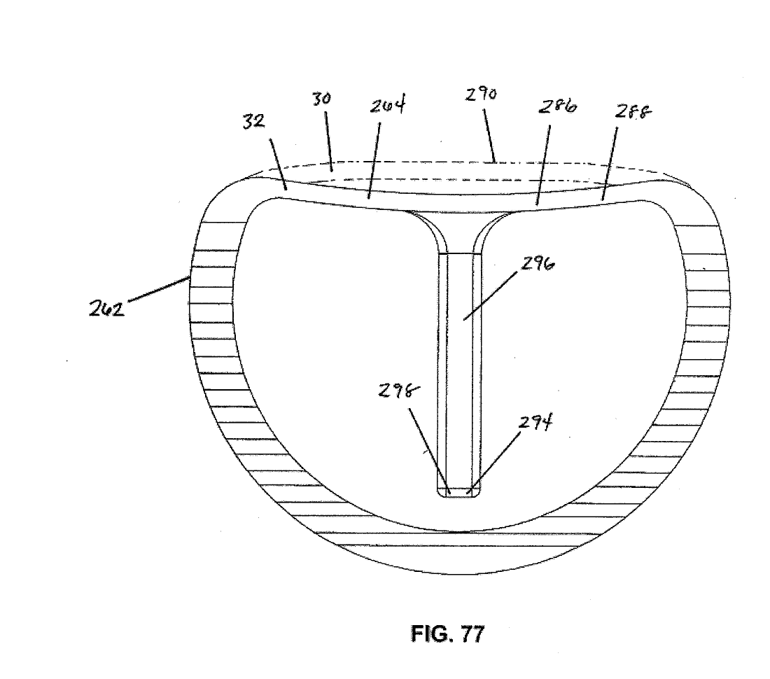

As shown in FIGS. 76-80, the ends 266 of the first extrusion member 262 are connected to the ends 274 of the second extrusion member 264 to form a wall 282 extending about an implant cavity 284. The implantation configuration 32 of the first and second implant extrusion members 262 and 264 is defined by the deformable wall 276 of the second implant extrusion member 264 oriented in a non-deformed state 286 such that the deformable wall 276 has a substantially straight configuration 288 extending between the rigid wall end portions 266. As shown in FIG. 77, the pre-implantation configuration 30 is defined by the deformable wall 276 having a substantially arcuate configuration 290 extending between the rigid wall ends 266.

The rigid wall portion 268 further includes at least one socket opening 292 therein for being engaged by a tool, such as discussed above. A distal end of the tool is configured to engage deformable wall 276 and urge the deformable wall 276 to deform from the implantation configuration 32 toward the pre-implantation configuration 30. Further, the tool is configured to selectively disengage the deformed deformable wall 276 thereby allowing the deformable wall 276 to return to its non-deformed configuration corresponding to the implantation configuration 32 of the first and second implant extrusion members 262 and 264.

The deformable wall portion 276 includes a tool engagement portion 294 for being engaged by a tool and being deformed to the pre-implantation configuration 30. As shown in FIGS. 76-78, the tool engagement portion 294 can include a cantilevered shaft 296 extending from the deformable wall 276 across the implant cavity 284 toward the rigid wall portion 268. The cantilevered shaft 296 includes a distal end 298 thereof for being engaged by a tool and urged away from the rigid wall portion 268 and toward the pre-implantation configuration 30. In the pre-implantation configuration 30, the bone graft material 34 packed into the implant cavity 284 is packed around the cantilevered shaft 296 and the portion of the tool extending into the implant cavity 284 and engaging the deformable wall tool engagement portion 294.

The second implant extrusion member 264 is formed of a material having elastic qualities thereby allowing the deformable wall 276 to be shifted between the implantation configuration 32 and the pre-implantation configuration 30. One exemplary material includes PEEK.

After the implant extrusion members 262 and 264 have been shifted from the pre-implantation configuration 30 to the implantation configuration 32, the socket opening 292 can be filled or otherwise patched. Alternatively, a plug member could be inserted therein, such as described in the third embodiment, resulting in more bone graft material 34 being extruded from the implant cavity 284. Further, socket openings 292 not engaged by the tool as the implant extrusion members 262 and 264 are shifted from the pre-implantation configuration 30 to the implantation configuration 32 can be patched or filled, such as with a patch or plug, prior to shifting of the first and second implant extrusion members 262 and 264 thereby obstructing any openings 292 in the walls 268 and 276 of the implant extrusion members 262 and 264 so that the extruded bone graft material 34 is extruded into the gaps 36 between the upper and lower vertebral engagement surfaces 270, 272, 278 and 280 and the bone endplates 6 and 8.

In a sixth embodiment, the first and second implant extrusion members 300 and 302 are connected to one another, with the first implant extrusion member 300 having a rigid wall portion 304 extending about a deformable wall portion 306 of the second implant extrusion member 302.

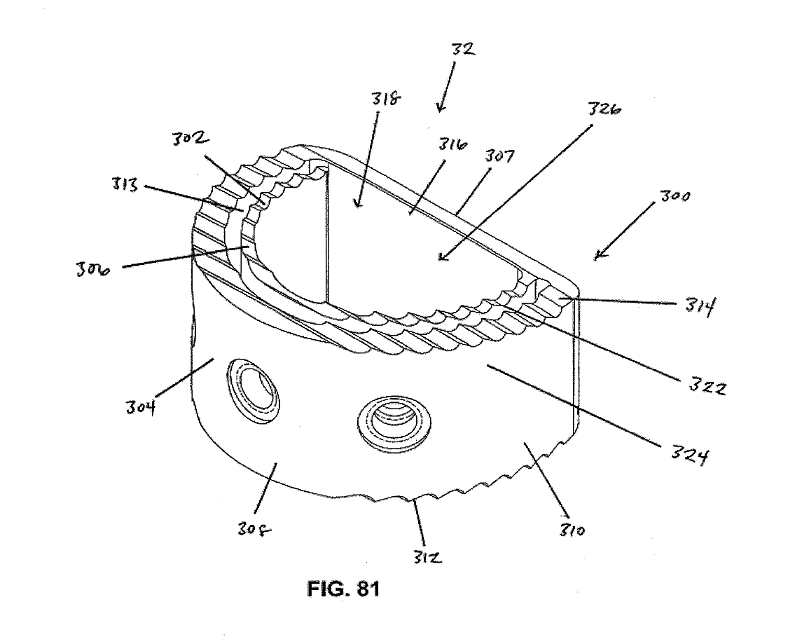

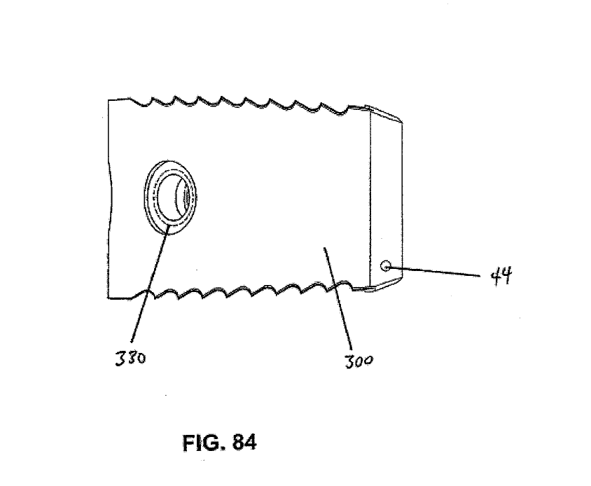

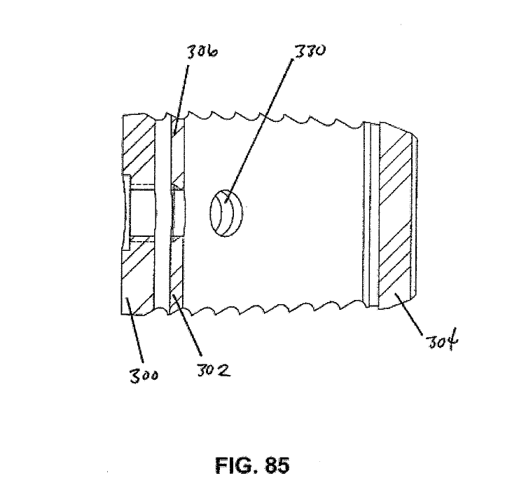

As shown in FIGS. 81-85, the first implant extrusion member 300 includes a rigid wall portion 304 includes a leading end portion 307, a trailing end portion 308 and lateral portions 310 extending therebetween. The rigid wall portion 304 includes upper and lower vertebral engaging surfaces 312 and 314. An inner surface 316 of the rigid wall portion 304 extends about a first cavity portion 318 of the first implant extrusion member 300.

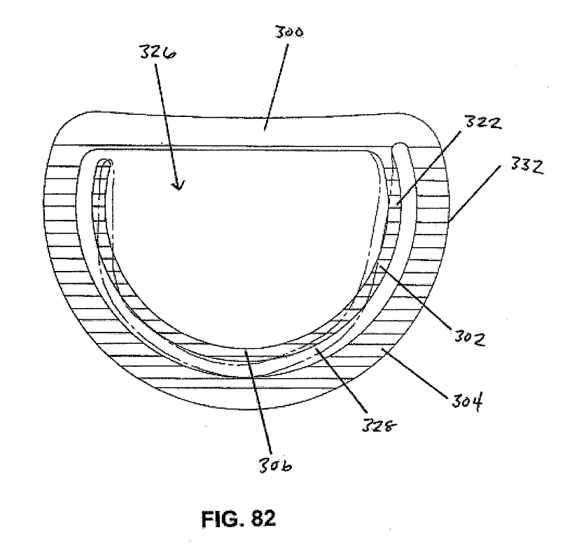

The deformable wall 306 of the second implant extrusion member 302 is connected to and extends from the inner surface 316 of the rigid wall 304 of the first implant extrusion member 300. As shown in FIG. 82, the deformable wall portion 306 extends from the leading wall 307 portion, along the lateral wall portions 310 and the trailing wall portion 308 such that a distal end 318 of the deformable wall portion 306 is adjacent to an opposite end 320 of the leading wall portion 307. Further, the deformable wall portion 306, in the non-deformed state 322, generally matches the curvature 324 of the adjacent rigid wall portion 304.

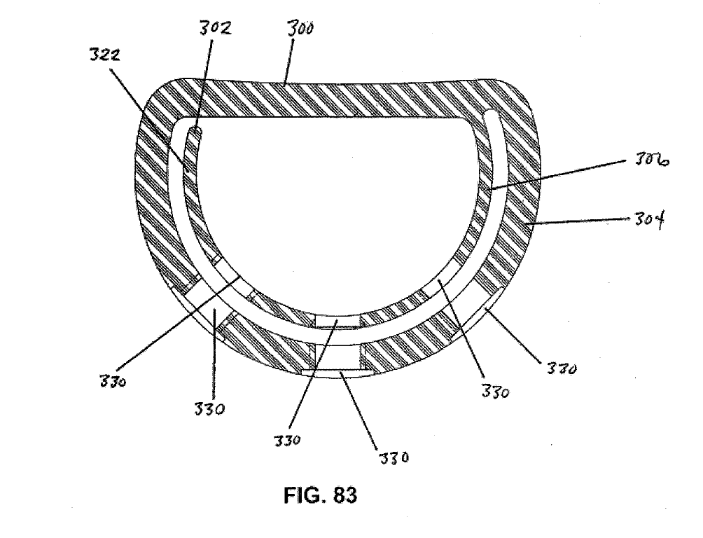

The deformable wall portion 306 and the rigid wall portion 304 cooperate to define an implant cavity 326 into which bone graft material 34 can be packed. The deformable wall portion 306 includes a non-deformed orientation 322, such as shown in FIGS. 81 and 83, corresponding to the implantation configuration 32 of the first and second implant extrusion members 300 and 302. Further, in a deformed orientation 328 of the deformable wall 306, the deformable wall 306 is drawn towards the rigid wall portion 302. As a result, the implant cavity 326 of the rigid and deformable wall portions 304 and 306 is enlarged and thus defines the pre-implantation configuration 30.

To engage and deform the deformable wall 306, both the deformable wall 306 and the rigid wall 304 include socket or tool openings 330 therein to be engaged by a tool such as shown in FIGS. 81 and 83. As discussed above, engagement of the tool with the socket openings 330 obstructs the openings 330 and prevents or restricts extrusion of material therethrough as the first and second implant extrusion members 300 and 302 shift from the pre-implantation 30 to the implantation configuration 32.

Prior to-placing the implant between vertebral bodies 6 and 8, the rigid and deformable walls 304 and 306 are engaged by the tool. The tool is operable to urge the deformable wall portion 306 toward the rigid wall 304 and into the deformed configuration 328 defining the pre-implantation configuration 30. With the tool still engaged, the implant is packed with bone graft material 34 and inserted between adjacent vertebrae 6 and 8. After the implant is then exposed to spinal loading the tool disengages the deformable wall portion 306 thereby allowing the deformable wall 306 to shift to the non-deformed orientation 322 defining the implantation configuration 32. As a result, bone graft material 34 is extruded from the cavity 326 into the space 36 between the upper and lower vertebral engaging surfaces 312 and 314 and the adjacent vertebral faces 6 and 8. The tool is then disengaged from the rigid wall 306, after which the tool openings 330 can be filled and/or the outer surface 332 of the implant can have bone graft material 34 applied thereto.

The second implant extrusion member 302 is formed of a material having elastic qualities thereby allowing the deformable wall 306 to be shifted between the implantation configuration 32 and the pre-implantation configuration 30. One exemplary material includes PEEK.

For any of the above described implants, prior to inserting the implant the surgical field and anesthetizing the patient are sterilized. A surgical incision is made in the patient from the anterior, or the front of the patient. An anterior approach is used because it provides greater blood flow to promote healing and causes less tissue damage than a posterior approach. Alternatively, a lateral approach from the side of the patient can be used based on the surgeon's preference. Once the incision is made the surrounding tissue is distracted or moved out of the way using standard instruments and distracting methodology.

The installation site of the implant is prepared by removing the severely damage tissue of the intervertebral disk, i.e. a discectomy. The disc can be removed with a ring curette which cuts out the disc. The endplates of the vertebra can then be roughened with the use of a rasp in order to remove all disc material and encourage blood flow and healing in the vertebral space. The roughening of the endplates also flattens the surface of the vertebrae to conform to the surface of the implant thus reducing the risk that the implant will shift out of position.

A tamp device tool or spacer can be inserted into the vertebral space until the correct implant size is determined. The size can be determined by inserting the smaller trial tamp devices and progressively increasing the size of the tamp device until the tamp device fits into the vertebral space. The color coding on the tamp device corresponds to the correct implant size and directs the selection of the correct implant size. Further description and drawings of the tamp device tool is available in U.S. Utility application Ser. No. 12/016,684 which is incorporated herein by reference.

As discussed above, bone graft material can be formed from synthetically made hydroxyapatite, synthetically made gelatin carrier, demineralized bone matrix, and the patient's own blood products and/or bone marrow extract to act as the bone void filler. However, other materials can be added to the bone void filler composition based on the surgeon's preference, such as bone morphogenic proteins (BMP). The bone void filler composition can be packed into the chambers, central bores, or voids of the implants. It is preferable to place equal masses of bone void filler in each of the implant chambers in order to prevent the implant from binding during collapse to the fully locked configuration or state. Furthermore, the placing of equal masses of bone void filler in each chamber results in the proper distribution of bone void filler to the endplate.

The surgeon can then attach the to an inserter or insertion tool. The exact attachment procedure is described above for each particular embodiment. The implant can be inserted into the vertebral space with or without the assistance of a distractor. A tamp or slap hammer may be required to adjust the position of the implant. The tamp or slap hammer can be directly applied by the surgeon to the particular implant.

Once the implant is in place the inserter or insertion tool can be removed causing the deployment of the bone void filler. The bone void filler is deployed with the attendant extrusion of bone void filler directly to the endplates of adjacent vertebrae. Deployment of the bone void filler can be achieved through a number of techniques depending on the implant, such as compression by the vertebral bodies.

Other implant embodiments rely on articulation of the insertion tool to deploy the bone void filler to the adjacent vertebrae. The exact deployment procedure is described above for each particular embodiment.

After the insertion of the implant a posterior approach can then used to implant a pedicle screw and rod system. During the implantation of the pedicle screw and rod system, a compressor can be used for compressing the vertebrae around the implant of the instant invention resulting in deployment of the instant invention. The 360-degree maneuver or circumferential fusion procedure can result in simultaneously fixing the vertebrae adjacent to the implant with a pedicle screw and rod system and deploying bone void filler.

Depending on surgeon's choice, a spinal plate can also be implanted to the vertebrae to provide supplemental fixation. The supplemental fixation serves the dual purposes of providing additional structural support to the vertebrae and to provide an obstruction to the implant should the implant be expelled from the vertebral space. However, it is intended that the implant be able to be implanted alone without the need of supplemental fixation. Note that the exact order of the steps in the surgical procedure can vary from surgeon to surgeon based on preference and from surgery to surgery based on the needs of the particular patient.

While there have been illustrated and described particular embodiments of the present invention, it will be appreciated that numerous changes and modifications will occur to those skilled in the art, and it is intended in the appended claims to cover all those changes and modifications which fall within the true spirit and scope of the present invention.

What is claimed is:

1. An implant for being inserted between adjacent vertebrae for promoting fusion therebetween, the implant comprising:
an implant body having upper and lower surfaces configured for facing corresponding upper and lower vertebrae;
a first implant extrusion member of the implant body;
a second implant extrusion member of the implant body;
a cavity of the implant body having an enlarged size with the first and second extrusion members in a pre-implantation configuration thereof for receiving bone graft material packed into the enlarged size cavity, the first and second extrusion members being configured to be shifted relative to one another during implantation to an implantation configuration with the cavity size being reduced from the pre-implantation enlarged size to extrude bone graft material out therefrom;
upper and lower openings of the cavity through which the bone graft material is extruded with the first and second implant members shifted from the pre-implantation configuration to the implantation configuration so that bone graft material is extruded out from the cavity through the upper and lower openings thereof to be disposed between the implant body upper and lower surfaces and corresponding facing vertebral surfaces; and
wall portions of the implant body that extend about the cavity to form the upper and lower openings thereof with the wall portions lacking unobstructed openings therethrough during shifting of the first and second implant extrusion members from the pre-implantation configuration to the implantation configuration so that bone graft material is only extruded out through the upper and lower cavity openings as the first and second members are shifted to the implantation configuration.

2. The implant of claim 1 wherein the first and second implant extrusion members have a ratchet connection therebetween for shifting the first and second implant members from the pre-implantation configuration to the implantation configuration.

3. The implant of claim 1 wherein the first implant extrusion member includes a central wall portion extending across the cavity and the second implant extrusion member includes a central sleeve portion extending across the cavity sized for fitting over and receiving the central wall portion of the first implant extrusion member therein and dividing the cavity into a pair of cavities.

4. The implant of claim 3 wherein the central wall portion and central sleeve portion have a ratchet connection therebetween.

5. The implant of claim 1 wherein the first implant extrusion member comprises a deformable wall portion of the implant body wall portions and the second extrusion member comprises a rigid wall portion of the implant body wall portions with the deformable wall portion being shifted relative to the rigid wall portion as the first and second implant extrusion members are shifted between pre-implantation and implantation configurations thereof.

6. The implant of claim 5 wherein the rigid wall portion extends about the deformable wall portion and the deformable wall portion shifts within the rigid wall portion.

7. The implant of claim 5 wherein the rigid wall portion has opposite ends and an arcuate configuration extending therebetween, and the deformable wall portion extends between the ends of the rigid wall portion and has a non-deformed configuration with the implant body in the implantation configuration and a deformed configuration with the implant body in the pre-implantation configuration.

8. The implant of claim 7 wherein the non-deformed configuration of the deformable wall portion is a substantially straight configuration of the deformable wall portion extending between the rigid wall portion opposite ends and the deformed configuration of the deformable wall portion is a substantially arcuate configuration of the deformable wall portion extending between the rigid wall portion opposite ends.

9. The implant of claim 1 wherein the wall portions include an opening for receiving the first implant extrusion member with the first implant extrusion member configured to be shifted through the opening into the implant cavity for shifting of the first and second implant extrusion members from the pre-implantation configuration to the implantation configuration.

10. The implant of claim 1 wherein the wall portions include a socket opening for receiving a corresponding portion of a tool therein, the tool portion received in the socket opening with the first and second implant extrusion members in the pre-implantation configuration and being operable to shift the extrusion members from the pre-implantation configuration to the implantation configuration.
